(19) United States
(12) Patent Application Publication
(10) Pub. No.: US 2011/0269788 A1
(43) Pub. Date: Nov. 3, 2011

(54) SPIRO-OXINDOLE-DERIVATIVES AS SODIUM CHANNEL BLOCKERS

(75) Inventors: Jean-Jacques Cadieux, Burnaby (CA); Mikhail Chafeev, Burnaby (CA); Sultan Chowdhury, Surrey (CA); Amy Frances Douglas, Vancouver (CA); Jianmin Fu, Coquitlam (CA); Jonathan Langille, Quaker Hill, CT (US); Shaoyi Sun, Coquitlam (CA); Mark Wood, Port Moody (CA)

(73) Assignee: Xenon Pharmaceuticals Inc., Burnaby (CA)

(21) Appl. No.: 13/142,375

(22) PCT Filed: Dec. 29, 2009
(86) PCT No.: PCT/US09/69663

§ 371 Date: Jun. 27, 2011

Related U.S. Application Data
(60) Provisional application No. 61/141,099, filed on Dec. 29, 2008.

(51) Int. Cl. A61K 31/4355; A61K 31/437; A61P 25/00; A61P 25/24; A61P 9/00; A61P 11/00; A61P 13/10; A61P 1/04; A61P 25/06; A61P 1/00; A61P 25/02; A61P 19/02; A61P 29/00; A61P 9/10; A61P 21/04; A61P 21/00; A61P 3/00; A61P 9/12; A61P 5/16; A61P 25/22; A61P 25/18; A61P 35/00; A61P 25/08; A61P 7/00; A61P 13/08; A61P 17/04; C07D 491/20

(52) U.S. Cl. 514/278; 546/15

(57) ABSTRACT

This invention is directed to spiro-oxindole compounds of formulas (I), (II), (III), as stereoisomers, enantiomers, tautomers thereof or mixtures thereof; or pharmaceutically acceptable salts, solvates or prodrugs thereof, for the treatment and/or prevention of sodium channel-mediated diseases or conditions, such as pain.

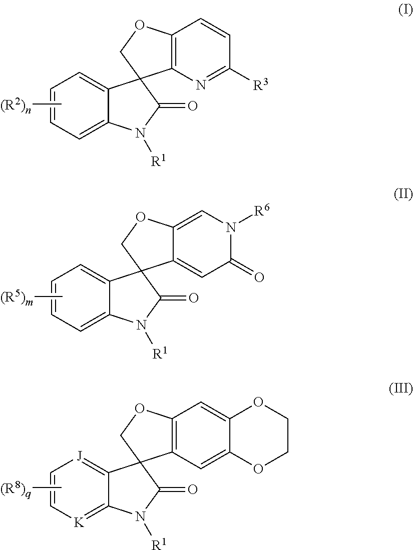

### FIELD OF THE INVENTION

The present invention is directed to spiro-oxindole compounds and pharmaceutical compositions comprising the compounds and methods of using the compounds and the pharmaceutical compositions in treating sodium channel-mediated diseases or conditions, such as pain, as well as other diseases and conditions associated with the mediation of sodium channels.

### BACKGROUND OF THE INVENTION

Voltage-gated sodium channels, transmembrane proteins that initiate action potentials in nerve, muscle and other electrically excitable cells, are a necessary component of normal sensation, emotions, thoughts and movements (Catterall, W. A., Nature (2001), Vol. 409, pp. 988-990). These channels consist of a highly processed alpha subunit that is associated with auxiliary beta subunits. The pore-forming alpha subunit is sufficient for channel function, but the kinetics and voltage dependence of channel gating are in part modified by the beta subunits (Goldin et al., Neuron (2000), Vol. 28, pp. 365-368). Each alpha-subunit contains four homologous domains, I to IV, each with six predicted transmembrane segments. The alpha-subunit of the sodium channel, forming the ion-conducting pore and containing the voltage sensors regulating sodium ion conduction has a relative molecular mass of 260,000. Electrophysiological recording, biochemical purification, and molecular cloning have identified ten different sodium channel alpha subunits and four beta subunits (Yu, F. H., et al., Sci. STKE (2004), 253; and Yu, F. H., et al., Neurosci. (2003), 20:7577-85).

The hallmarks of sodium channels include rapid activation and inactivation when the voltage across the plasma membrane of an excitable cell is depolarized (voltage-dependent gating), and efficient and selective conduction of sodium ions through conducting pores intrinsic to the structure of the protein (Sato, C., et al., Nature (2001), 409:1047-1051). At negative or hyperpolarized membrane potentials, sodium channels are closed. Following membrane depolarization, sodium channels open rapidly and then inactivate. Channels only conduct currents in the open state and, once inactivated, have to return to the resting state, favoured by membrane hyperpolarization, before they can reopen. Different sodium channel subtypes vary in the voltage range over which they activate and inactivate as well as their activation and inactivation kinetics.

The sodium channel family of proteins has been extensively studied and shown to be involved in a number of vital body functions. Research in this area has identified variants of the alpha subunits that result in major changes in channel function and activities, which can ultimately lead to major pathophysiological conditions. Implicit with function, this family of proteins are considered prime points of therapeutic intervention. Naᵥ1.1 and Naᵥ1.2 are highly expressed in the brain (Raymond, C. K., et al., J. Biol. Chem. (2004), 279(44):46234-41) and are vital to normal brain function. In humans, mutations in Naᵥ1.1 and Naᵥ1.2 result in severe epileptic states and in some cases mental decline (Rhodes, T. H., et al., Proc. Natl. Acad. Sci. USA (2004), 101(30):11147-52; Kamiya, K., et al., J. Biol. Chem. (2004), 24(11):2690-8; Pereira, S., et al., Neurology (2004), 63(1):191-2). As such both channels have been considered as validated targets for the treatment of epilepsy (see PCT Published Patent Publication No. WO 01/38564).

Naᵥ1.3 is broadly expressed throughout the body (Raymond, C. K., et al., op. cit.). It has been demonstrated to have its expression upregulated in the dorsal horn sensory neurons of rats after nervous system injury (Hains, B. D., et al., J. Neurosci. (2003), 23(26):8881-92). Many experts in the field have considered Naᵥ1.3 as a suitable target for pain therapeutics (Lai, J., et al., Curr. Opin. Neurobiol. (2003), (3):291-72003; Wood, J. N., et al., J. Neurobiol. (2004), 61(1):55-71; Chung, J. M., et al., Novartis Found Symp. (2004), 261:19-27; discussion 27-31, 47-54).

Naᵥ1.4 expression is essentially limited to muscle (Raymond, C. K., et al., op. cit.). Mutations in this gene have been shown to have profound effects on muscle function including paralysis, (Tamaoka A., Intern. Med. (2003), (9):769-70). Thus, this channel can be considered a target for the treatment of abnormal muscle contractility, spasm or paralysis.

The cardiac sodium channel, Naᵥ1.5, is expressed mainly in the heart ventricles and atria (Raymond, C. K., et al., op. cit.), and can be found in the sinovial node, ventricular node and possibly Purkinje cells. The rapid upstroke of the cardiac action potential and the rapid impulse conduction through cardiac tissue is due to the opening of Naᵥ1.5. As such, Naᵥ1.5 is central to the genesis of cardiac arrhythmias. Mutations in human Naᵥ1.5 result in multiple arrhythmic syndromes, including, for example, long QT3 (LQT3), Brugada syndrome (BS), an inherited cardiac conduction defect, sudden unexpected nocturnal death syndrome (SUNDS) and sudden infant death syndrome (SIDS) (Liu, H. et al., Am. J. Pharmacogenomics (2003), 3(3):173-9). Sodium channel blocker therapy has been used extensively in treating cardiac arrhythmias. The first antiarrhythmic drug, quinidine, discovered in 1914, is classified as a sodium channel blocker.

Naᵥ1.6 encodes an abundant, widely distributed voltage-gated sodium channel found throughout the central and peripheral nervous systems, clustered in the nodes of Ranvier of neural axons (Caldwell, J. H., et al., Proc. Natl. Acad. Sci. USA (2000), 97(10): 5616-20). Although no mutations in humans have been detected, Naᵥ1.6 is thought to play a role in the manifestation of the symptoms associated with multiple sclerosis and has been considered as a target for the treatment of this disease (Craner, M. J., et al., Proc. Natl. Acad. Sci. USA (2004), 101(21):8168-73).

Naᵥ1.7 was first cloned from the pheochromocytoma PC12 cell line (Toledo-Aral, J. J., et al., Proc. Natl. Acad. Sci. USA (1997), 94:1527-1532). Its presence at high levels in the growth cones of small-diameter neurons suggested that it could play a role in the transmission of nociceptive information. Although this has been challenged by experts in the field as Naᵥ1.7 is also expressed in neuroendocrine cells associated with the autonomic system (Klugbauer, N., et al., EMBO J. (1995), 14(6):1084-90) and as such has been implicated in autonomic processes. The implicit role in autonomic functions was demonstrated with the generation of Naᵥ1.7 null mutants; deleting Naᵥ1.7 in all sensory and sympathetic neurons resulted in a lethal perinatal phenotype. (Nassar, et al., Proc. Natl. Acad. Sci. USA (2004), 101(34):12706-11). In contrast, by deleting the Naᵥ1.7 expression in a subset of sensory neurons that are predominantly nociceptive, a role in pain mechanisms, was demonstrated (Nassar, et al., op. cit.). Further support for Naᵥ1.7 blockers active in a subset of neurons is supported by the finding that two human heritable pain conditions, primary erythermalgia and familial rectal pain, have been shown to map to Naᵥ1.7 (Yang, Y., et al., J. Med. Genet. (2004), 41(3):171-4).

The expression of Naᵥ1.8 is essentially restricted to the DRG (Raymond, C. K., et al., op. cit.). There are no identified human mutations for Naᵥ1.8. However, Naᵥ1.8-null mutant mice were viable, fertile and normal in appearance. A pronounced analgesia to noxious mechanical stimuli, small deficits in noxious thermoreception and delayed development of inflammatory hyperalgesia suggested to the researchers that Naᵥ1.8 plays a major role in pain signalling (Akopian, A. N., et al., Nat. Neurosci. (1999), 2(6): 541-8). Blocking of this channel is widely accepted as a potential treatment for pain (Lai, J, et al., op. cit.; Wood, J. N., et al., op. cit.; Chung, J. M., et al., op. cit.). PCT Published Patent Application No. WO03/037274A2 describes pyrazole-amides and sulfonamides for the treatment of central or peripheral nervous system conditions, particularly pain and chronic pain by blocking sodium channels associated with the onset or recurrence of the indicated conditions. PCT Published Patent Application No. WO 03/037890A2 describes piperidines for the treatment of central or peripheral nervous system conditions, particularly pain and chronic pain by blocking sodium channels associated with the onset or recurrence of the indicated conditions. The compounds, compositions and methods of these inventions are of particular use for treating neuropathic or inflammatory pain by the inhibition of ion flux through a channel that includes a PN3 (Naᵥ1.8) subunit.

The tetrodotoxin insensitive, peripheral sodium channel Naᵥ1.9, disclosed by Dib-Hajj, S. D., et al. (see Dib-Hajj, S. D., et al., Proc. Natl. Acad. Sci. USA (1998), 95(15):8963-8) was shown to reside solely in the dorsal root ganglia. It has been demonstrated that Naᵥ1.9 underlies neurotrophin (BDNF)-evoked depolarization and excitation, and is the only member of the voltage gated sodium channel superfamily to be shown to be ligand mediated (Blum, R., Kafitz, K. W., Konnerth, A., Nature (2002), 419 (6908):687-93). The limited pattern of expression of this channel has made it a candidate target for the treatment of pain (Lai, J, et al., op. cit.; Wood, J. N., et al., op. cit.; Chung, J. M. et al., op. cit.).

NaX is a putative sodium channel, which has not been shown to be voltage gated. In addition to expression in the lung, heart, dorsal root ganglia, and Schwann cells of the peripheral nervous system, NaX is found in neurons and ependymal cells in restricted areas of the CNS, particularly in the circumventricular organs, which are involved in body-fluid homeostasis (Watanabe, E., et al., J. Neurosci. (2000), 20(20):7743-51). NaX-null mice showed abnormal intakes of hypertonic saline under both water- and salt-depleted conditions. These findings suggest that the NaX plays an important role in the central sensing of body-fluid sodium level and regulation of salt intake behaviour. Its pattern of expression and function suggest it as a target for the treatment of cystic fibrosis and other related salt regulating maladies.

Studies with the sodium channel blocker tetrodotoxin (TTX) used to lower neuron activity in certain regions of the brain, indicate its potential use in the treatment of addiction. Drug-paired stimuli elicit drug craving and relapse in addicts and drug-seeking behaviour in rats. The functional integrity of the basolateral amygdala (BLA) is necessary for reinstatement of cocaine-seeking behaviour elicited by cocaine-conditioned stimuli, but not by cocaine itself. BLA plays a similar role in reinstatement of heroin-seeking behavior. TTX-induced inactivation of the BLA on conditioned and heroin-primed reinstatement of extinguished heroin-seeking behaviour in a rat model (Fuchs, R. A. and See, R. E., Psychopharmacology (2002), 160(4):425-33).

This closely related family of proteins has long been recognised as targets for therapeutic intervention. Sodium channels are targeted by a diverse array of pharmacological agents. These include neurotoxins, antiarrhythmics, anticonvulsants and local anesthetics (Clare, J. J., et al., Drug Discovery Today (2000), 5:506-520). All of the current pharmacological agents that act on sodium channels have receptor sites on the alpha subunits. At least six distinct receptor sites for neurotoxins and one receptor site for local anesthetics and related drugs have been identified (Cestèle, S. et al., Biochimie (2000), Vol. 82, pp. 883-892).

The small molecule sodium channel blockers or the local anesthetics and related antiepileptic and antiarrhythmic drugs, interact with overlapping receptor sites located in the inner cavity of the pore of the sodium channel (Catterall, W. A., Neuron (2000), 26:13-25). Amino acid residues in the S6 segments from at least three of the four domains contribute to this complex drug receptor site, with the IVS6 segment playing the dominant role. These regions are highly conserved and as such most sodium channel blockers known to date interact with similar potency with all channel subtypes. Nevertheless, it has been possible to produce sodium channel blockers with therapeutic selectivity and a sufficient therapeutic window for the treatment of epilepsy (e.g. lamotrignine, phenytoin and carbamazepine) and certain cardiac arrhythmias (e.g. lignocaine, tocainide and mexiletine). However, the potency and therapeutic index of these blockers is not optimal and have limited the usefulness of these compounds in a variety of therapeutic areas where a sodium channel blocker would be ideally suited.

### Management of Acute and Chronic Pain

Drug therapy is the mainstay of management for acute and chronic pain in all age groups, including neonates, infants and children. The pain drugs are classified by the American Pain Society into three main categories: 1) non-opioid analgesics-acetaminophen, and non-steroidal anti-inflammatory drugs (NSAIDs), including salicylates (e.g. aspirin), 2) opioid analgesics and 3) co-analgesics.

Non-opioid analgesics such as acetaminophen and NSAIDs are useful for acute and chronic pain due to a variety of causes including surgery, trauma, arthritis and cancer. NSAIDs are indicated for pain involving inflammation because acetaminophen lacks anti-inflammatory activity. Opioids also lack anti-inflammatory activity. All NSAIDs inhibit the enzyme cyclooxygenase (COX), thereby inhibiting prostaglandin synthesis and reducing the inflammatory pain response. There are at least two COX isoforms, COX-1 and COX-2. Common non-selective COX inhibitors include, ibuprofen and naproxen. Inhibition of COX-1, which is found in platelets, GI tract, kidneys and most other human tissues, is thought to be associated with adverse effects such as gastrointestinal bleeding. The development of selective COX-2 NSAIDs, such as Celecoxib, Valdecoxib and Rofecoxib, have the benefits of non-selective NSAIDs with reduced adverse effect profiles in the gut and kidney. However, evidence now suggests that chronic use of certain selective COX-2 inhibitors can result in an increased risk of stroke occurrence.

The use of opioid analgesics is recommended by the American Pain Society to be initiated based on a pain-directed history and physical that includes repeated pain assessment. Due to the broad adverse effect profiles associated with opiate use, therapy should include a diagnosis, integrated interdisciplinary treatment plan and appropriate ongoing patient monitoring. It is further recommended that opioids be added to non-opioids to manage acute pain and cancer related pain that does not respond to non-opioids alone. Opioid analgesics act as agonists to specific receptors of the mu and kappa types in the central and peripheral nervous system. Depending on the opioid and its formulation or mode of administration it can be of shorter or longer duration. All opioid analgesics have a risk of causing respiratory depression, liver failure, addiction and dependency, and as such are not ideal for long-term or chronic pain management.

A number of other classes of drugs may enhance the effects of opioids or NSAIDSs, have independent analgesic activity in certain situations, or counteract the side effects of analgesics. Regardless of which of these actions the drug has, they are collectively termed “coanalgesics”. Tricyclic antidepressants, antiepileptic drugs, local anaesthetics, glucocorticoids, skeletal muscle relaxants, anti-spasmodil agents, antihistamines, benzodiazepines, caffeine, topical agents (e.g. capsaicin), dextroamphetamine and phenothizines are all used in the clinic as adjuvant therapies or individually in the treatment of pain. The antiepeileptic drugs in particular have enjoyed some success in treating pain conditions. For instance, Gabapentin, which has an unconfirmed therapeutic target, is indicated for neuropathic pain. Other clinical trials are attempting to establish that central neuropathic pain may respond to ion channel blockers such as blockers of calcium, sodium and/or NMDA (N-methyl-D-aspartate) channels. Currently in development are low affinity NMDA channel blocking agents for the treatment of neuropathic pain. The literature provides substantial pre-clinical electrophysiological evidence in support of the use of NMDA antagonists in the treatment of neuropathic pain. Such agents also may find use in the control of pain after tolerance to opioid analgesia occurs, particularly in cancer patients.

Systemic analgesics such as NSAIDs and opioids are to be distinguished from therapeutic agents which are useful only as local analgesics/anaesthetics. Well known local analgesics such as lidocaine and xylocaine are non-selective ion channel blockers which can be fatal when administered systemically. A good description of non-selective sodium channel blockers is found in Madge, D. et al., J. Med. Chem. (2001), 44(2):115-37.

Several sodium channel modulators are known for use as anticonvulsants or antidepressants, such as carbamazepine, amitriptyline, lamotrigine and riluzole, all of which target brain tetradotoxin-sensitive (TTX-S) sodium channels. Such TTX-S agents suffer from dose-limiting side effects, including dizziness, ataxia and somnolence, primarily due to action at TTX-S channels in the brain.

### Sodium Channels Role in Pain

Sodium channels play a diverse set of roles in maintaining normal and pathological states, including the long recognized role that voltage gated sodium channels play in the generation of abnormal neuronal activity and neuropathic or pathological pain (Chung, J. M. et al., op. cit.). Damage to peripheral nerves following trauma or disease can result in changes to sodium channel activity and the development of abnormal afferent activity including ectopic discharges from axotomised afferents and spontaneous activity of sensitized intact nociceptors. These changes can produce long-lasting abnormal hypersensitivity to normally innocuous stimuli, or allodynia. Examples of neuropathic pain include, but are not limited to, post-herpetic neuralgia, trigeminal neuralgia, diabetic neuropathy, chronic lower back pain, phantom limb pain, and pain resulting from cancer and chemotherapy, chronic pelvic pain, complex regional pain syndrome and related neuralgias.

There has been some degree of success in treating neuropathic pain symptoms by using medications, such as gabapentin, and more recently pregabalin, as short-term, first-line treatments. However, pharmacotherapy for neuropathic pain has generally had limited success with little response to commonly used pain reducing drugs, such as NSAIDS and opiates. Consequently, there is still a considerable need to explore novel treatment modalities.

There remains a limited number of potent effective sodium channel blockers with a minimum of adverse events in the clinic. There is also an unmet medical need to treat neuropathic pain and other sodium channel associated pathological states effectively and without adverse side effects. The present invention provides methods to meet these critical needs.

### SUMMARY OF THE INVENTION

The present invention is directed to spiro-oxindole compounds and pharmaceutical compositions comprising the compounds and methods of using the compounds and the pharmaceutical compositions of the invention for the treatment and/or prevention of sodium channel-mediated diseases or conditions, such as pain. The present invention is also directed to methods of using the compounds of the invention and pharmaceutical compositions comprising the compounds of the invention for the treatment of other sodium channel-mediated diseases or conditions, including, but not limited to central nervous conditions such as epilepsy, anxiety, depression and bipolar disease; cardiovascular conditions such as arrhythmias, atrial fibrillation and ventricular fibrillation; neuromuscular conditions such as restless leg syndrome, essential tremour and muscle paralysis or tetanus; neuroprotection against stroke, glaucoma, neural trauma and multiple sclerosis; and channelopathies such as erythromyalgia and familial rectal pain syndrome. The present invention is also directed to the use of the compounds of the invention and pharmaceutical compositions comprising the compounds of the invention for the treatment and/or prevention of diseases or conditions, such as hypercholesterolemia, benign prostatic hyperplasia, pruritis, and cancer.

Accordingly, in one aspect, the invention is directed a compound of formula (I):

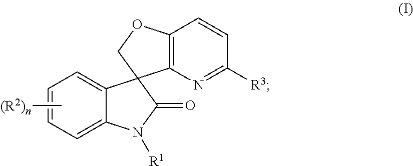

wherein:
n is 1 or 2;
R¹ is [3-(trifluoromethyl)pyridin-2-yl]methyl, tetrahydrofuran-2-ylmethyl, (2R)-tetrahydrofuran-2-ylmethyl, (2S)-tetrahydrofuran-2-ylmethyl or 2,3-dihydro-1,4-benzodioxin-6-ylmethyl;
each R² is independently selected from hydrogen or halo; and
R³ is methoxy, ethoxy or halo;
as a stereoisomer, enantiomer, tautomer thereof or mixtures thereof;
or a pharmaceutically acceptable salt, solvate or prodrug thereof.

In another aspect, this invention is directed to compounds of formula (II):

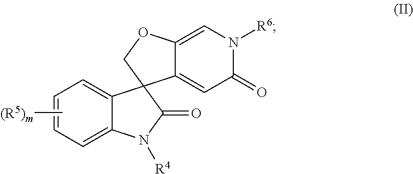

wherein:
m is 1 or 2;
R⁴ is [3-(trifluoromethyl)pyridin-2-yl]methyl, tetrahydrofuran-2-ylmethyl, (2R)-tetrahydrofuran-2-ylmethyl or (2S)-tetrahydrofuran-2-ylmethyl;
each R⁵ is independently selected from hydrogen or halo; and
R⁶ is hydrogen or alkyl;
as a stereoisomer, enantiomer, tautomer thereof or mixtures thereof;
or a pharmaceutically acceptable salt, solvate or prodrug thereof.

In another aspect, this invention is directed to compounds of formula (III):

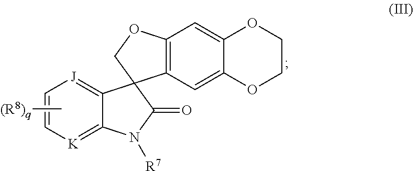

wherein:
q is 1 or 2;
one of J and K is —N═ and the other is —C(R⁸)═;
R⁷ is hydrogen, diphenylmethyl, pyridin-2-ylmethyl or [3-(trifluoromethyl)pyridin-2-yl]methyl; and
each R⁸ is selected from hydrogen or halo;
as a stereoisomer, enantiomer, tautomer thereof or mixtures thereof;
or a pharmaceutically acceptable salt, solvate or prodrug thereof.

In another aspect, the invention is directed to pharmaceutical compositions comprising a pharmaceutically acceptable excipient and a therapeutically effective amount of a compound of the invention, as set forth above, as a stereoisomer, enantiomer, tautomer thereof or mixtures thereof; or as a pharmaceutically acceptable salt, solvate or prodrug thereof.

In another aspect, the invention provides methods for the treatment of pain in a mammal, preferably a human, wherein the methods comprise administering to the mammal in need thereof a therapeutically effective amount of a compound of the invention, as set forth above, as a stereoisomer, enantiomer, tautomer thereof or mixtures thereof; or a pharmaceutically acceptable salt, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the present invention provides a method for treating or lessening the severity of a disease, condition, or disorder in a mammal where activation or hyperactivity of one or more of Naᵥ1.1, Naᵥ1.2, Naᵥ1.3, Naᵥ1.4, Naᵥ1.5, Naᵥ1.6, Naᵥ1.7, Naᵥ1.8, or Naᵥ1.9 is implicated in the disease, condition or disorder, wherein the method comprises administering to the mammal in need thereof a therapeutically effective amount of a compound of the invention, as set forth above, as a stereoisomer, enantiomer, tautomer thereof or mixtures thereof; or a pharmaceutically acceptable salt, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides methods of treating a range of sodium channel-mediated diseases or conditions in a mammal, for example, pain associated with HIV, HIV treatment induced neuropathy, trigeminal neuralgia, post-herpetic neuralgia, eudynia, heat sensitivity, tosarcoidosis, irritable bowel syndrome, Crohns disease, pain associated with multiple sclerosis (MS), amyotrophic lateral sclerosis (ALS), diabetic neuropathy, peripheral neuropathy, arthritic, rheumatoid arthritis, osteoarthritis, atherosclerosis, paroxysmal dystonia, myasthenia syndromes, myotonia, malignant hyperthermia, cystic fibrosis, pseudoaldosteronism, rhabdomyolysis, hypothyroidism, bipolar depression, anxiety, schizophrenia, sodium channel toxin related illnesses, familial erythermalgia, primary erythermalgia, familial rectal pain, cancer, epilepsy, partial and general tonic seizures, restless leg syndrome, arrhythmias, fibromyalgia, neuroprotection under ischaemic conditions caused by stroke, glaucoma or neural trauma, tachy-arrhythmias, atrial fibrillation and ventricular fibrillation, wherein the methods comprise administering to the mammal in need thereof, preferably a human, a therapeutically effective amount of a compound of the invention, as set forth above, as a stereoisomer, enantiomer, tautomer thereof or mixtures thereof, or a pharmaceutically acceptable salt, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides methods of treating a range of sodium channel-mediated diseases or conditions in a mammal, preferably a human, by the inhibition of ion flux through a voltage-dependent sodium channel in the mammal, wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides methods of treating or preventing hypercholesterolemia in a mammal, preferably a human, wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides methods of treating or preventing benign prostatic hyperplasia in a mammal, preferably a human, wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides methods of treating or preventing pruritis in a mammal, preferably a human, wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides methods of treating or preventing cancer in a mammal, preferably a human, wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides pharmaceutical therapy in combination with one or more other compounds of the invention or one or more other accepted therapies or as any combination thereof to increase the potency of an existing or future drug therapy or to decrease the adverse events associated with the accepted therapy. In one embodiment, the present invention relates to a pharmaceutical composition combining compounds of the present invention with established or future therapies for the indications listed in the invention.

In another aspect, this invention is directed to the use of the compounds of the invention, as set forth above, as a stereoisomer, enantiomer, tautomer thereof or mixtures thereof, or a pharmaceutically acceptable salt, solvate or prodrug thereof, or the use of a pharmaceutical composition comprising a pharmaceutically acceptable excipient and a compound of the invention, as set forth above, as a stereoisomer, enantiomer, tautomer thereof or mixtures thereof, or a pharmaceutically acceptable salt, solvate or prodrug thereof, in the preparation of a medicament for the treatment of sodium channel-mediated diseases or conditions in a mammal.

### DETAILED DESCRIPTION OF THE INVENTION

### Definitions

Certain chemical groups named herein may be preceded by a shorthand notation indicating the total number of carbon atoms that are to be found in the indicated chemical group. For example; C₇-C₁₂alkyl describes an alkyl group, as defined below, having a total of 7 to 12 carbon atoms, and C₄-C₁₂cycloalkylalkyl describes a cycloalkylalkyl group, as defined below, having a total of 4 to 12 carbon atoms. The total number of carbons in the shorthand notation does not include carbons that may exist in substituents of the group described.

In addition to the foregoing, as used in the specification and appended claims, unless specified to the contrary, the following terms have the meaning indicated:

“Alkyl” refers to a straight or branched hydrocarbon chain radical consisting solely of carbon and hydrogen atoms, containing no unsaturation, having from one to twelve carbon atoms, preferably one to eight carbon atoms or one to six carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, and the like.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like. The alkyl part of the haloalkyl radical may be optionally substituted as defined above for an alkyl group.

“Analgesia” refers to an absence of pain in response to a stimulus that would normally be painful.

“Allodynia” refers to a condition in which a normally innocuous sensation, such as pressure or light touch, is perceived as being extremely painful.

“Prodrugs” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., “Pro-drugs as Novel Delivery Systems,” A.C.S. Symposium Series, Vol. 14, and in Bioreversible Carriers in Drug Design, Ed. Edward B. Roche, American Pharmaceutical Association and Pergamon Press, 1987, both of which are incorporated in full by reference herein.

The term “prodrug” is also meant to include any covalently bonded carriers, which release the active compound of the invention in vivo when such prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in the compounds of the invention and the like.

The invention disclosed herein is also meant to encompass all pharmaceutically acceptable compounds of the invention being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabelled compounds could be useful to help determine or measure the effectiveness of the compounds, by characterizing, for example, the site or mode of action on the sodium channels, or binding affinity to pharmacologically important site of action on the sodium channels. Certain isotopically-labelled compounds of the invention, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of the invention can generally be prepared by conventional techniques known to those skilled in the art or by processes analogous to those described in the Preparations and Examples as set out below using an appropriate isotopically-labeled reagent in place of the non-labeled reagent previously employed.

The invention disclosed herein is also meant to encompass the in vivo metabolic products of the disclosed compounds. Such products may result from, for example, the oxidation, reducation, hydrolysis, amidation, esterification, and the like of the administered compound, primarily due to enzymatic processes. Accordingly, the invention includes compounds produced by a process comprising contacting a compound of this invention with a mammal for a period of time sufficient to yield a metabolic product thereof. Such products are typically are identified by administering a radiolabelled compound of the invention in a detectable dose to an animal, such as rat, mouse, guinea pig, monkey, or to human, allowing sufficient time for metabolism to occur, and isolating its coversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets, (e.g., cats, dogs, swine, cattle, sheep, goats, horses, rabbits), and non-domestic animals such as wildelife and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution. When a functional group is described as “optionally substituted,” and in turn, substitutents on the functional group are also “optionally substituted” and so on, for the purposes of this invention, such iterations are limited to five.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the invention. As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“Therapeutically effective amount” refers to that amount of a compound of the invention which, when administered to a mammal, preferably a human, is sufficient to effect treatment, as defined below, of a sodium channel-mediated disease or condition in the mammal, preferably a human. The amount of a compound of the invention which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:

(a) preventing the disease or condition from occurring in a mammal, in particular, when such mammal is predisposed to the condition but has not yet been diagnosed as having it;

(b) inhibiting the disease or condition, i.e., arresting its development;

(c) relieving the disease or condition, i.e., causing regression of the disease or condition; or

(d) relieving the symptoms resulting from the disease or condition, i.e., relieving pain without addressing the underlying disease or condition.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the invention, or their pharmaceutically acceptable salts may contain one or more asymmetric centres and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallisation. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present invention includes tautomers of any said compounds.

Also within the scope of the invention are all polymorphs of the compounds of the invention and crystal habits thereof.

The chemical naming protocol and structure diagrams used herein are a modified form of the I.U.P.A.C. nomenclature system, using the ACD/Name Version 9.07 software program, wherein the compounds of the invention are named herein as derivatives of a central core structure. For complex chemical names employed herein, a substituent group is named before the group to which it attaches. For example, cyclopropylethyl comprises an ethyl backbone with cyclopropyl substituent. In chemical structure diagrams, all bonds are identified, except for some carbon atoms, which are assumed to be bonded to sufficient hydrogen atoms to complete the valency.

Thus, for example, a compound of formula (I), as set forth above in the Summary of the Invention, wherein n is 1, R¹ is (2R)-tetrahydrofuran-2-ylmethyl, R² is bromo and R³ is methoxy; i.e., a compound of the following formula:

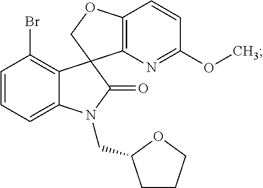

is named herein as 4′-bromo-5-methoxy-t-[(2R)-tetrahydrofuran-2-ylmethyl]spiro[furo[3,2-b]pyridine-3,3′-indol]-2′(1′H)-one.

Unless the context requires otherwise, throughout the specification and claims which follow, the transitional phrases “comprise” and variations thereof, such as, “comprises”, “comprising”, “comprising of” and “comprised of” are to be construed to be synonymous with “including”, “containing” or “characterized by” and are to be construed to be inclusive and open-ended in that additional, unrecited elements or method steps are not excluded.

### Embodiments of the Invention

Of the compounds of formula (I), as described above in the Summary of the Invention, preferred embodiments are selected from:
4′-bromo-5-methoxy-1′-[(2R)-tetrahydrofuran-2-ylmethyl]spiro[furo[3,2-b]pyridine-3,3′-indol]-2′(1′H)-one;
5-methoxy-1′-[(2R)-tetrahydrofuran-2-ylmethyl]spiro[furo[3,2-b]pyridine-3,3′-indol]-2′(1′H)-one;
5-methoxy-1′-{[3-(trifluoromethyl)pyridin-2-yl]methyl}spiro[furo[3,2-b]pyridine-3,3′-indol]-2′(1′H)-one; or
1′-(2,3-dihydro-1,4-benzodioxin-6-ylmethyl)-5-methoxyspiro[furo[3,2-b]pyridine-3,3′-indol]-2′(1H)-one.

Of the compounds of formula (II), as described above in the Summary of the Invention, a preferred embodiment is 1′-[(2R)-tetrahydrofuran-2-ylmethyl]spiro[furo[2,3-c]pyridine-3,3′-indole]-2′,5(1′H,6H)-dione.

Of the compounds of formula (III), as described above in the Summary of the Invention, a preferred embodiment is where J is —C(R⁸)═ and K is —N═. Another preferred embodiment is where J is —N═ and K is —C(R⁸)═. Another preferred embodiment is a compound selected from:
1′-(diphenylmethyl)-2,3-dihydrospiro[furo[2,3-g][1,4]benzodioxine-8,3′-pyrrolo[3,2-b]pyridin]-2′(1′H)-one;
2,3-dihydrospiro[furo[2,3-g][1,4]benzodioxine-8,3′-pyrrolo[3,2-b]pyridin]-2′(1′H)-one;
1′-(pyridin-2-ylmethyl)-2,3-dihydrospiro[furo[2,3-g][1,4]benzodioxine-8,3′-pyrrolo[3,2-b]pyridin]-2′(1′H)-one;
1′-{[3-(trifluoromethyl)pyridin-2-yl]methyl}-2,3-dihydrospiro[furo[2,3-g][1,4]benzodioxine-8,3′-pyrrolo[3,2-b]pyridin]-2′(1′H)-one;
1′-(diphenylmethyl)-2,3-dihydrospiro[furo[2,3-g][1,4]benzodioxine-8,3′-pyrrolo[2,3-b]pyridin]-2′(1′H)-one;
2,3-dihydrospiro[furo[2,3-g][1,4]benzodioxine-8,3′-pyrrolo[2,3-b]pyridin]-2′(1′H)-one;
1′-(pyridin-2-ylmethyl)-2,3-dihydrospiro[furo[2,3-g][1,4]benzodioxine-8,3′-pyrrolo[2,3-b]pyridin]-2′(1′H)-one; or
1′-{[3-(trifluoromethyl)pyridin-2-yl]methyl}-2,3-dihydrospiro[furo[2,3-g][1,4]benzodioxine-8,3′-pyrrolo[2,3-b]pyridin]-2′(1′H)-one.

Another embodiment of the invention is a method of treating, preventing or ameliorating a disease or a condition in a mammal, preferably a human, wherein the disease or condition is selected from the group consisting of pain, depression, cardiovascular diseases, respiratory diseases, and psychiatric diseases, and combinations thereof, and wherein the method comprises administering to the mammal in need thereof a therapeutically effective amount of an embodiment of a compound of the invention, as set forth above, as a stereoisomer, enantiomer, tautomer thereof or mixtures thereof, or a pharmaceutically acceptable salt, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

One embodiment of this embodiment is wherein the disease or condition is selected from the group consisting of neuropathic pain, inflammatory pain, visceral pain, cancer pain, chemotherapy pain, trauma pain, surgical pain, post-surgical pain, childbirth pain, labor pain, neurogenic bladder, ulcerative colitis, chronic pain, dental pain, persistent pain, peripherally mediated pain, centrally mediated pain, chronic headache, migraine headache, sinus headache, tension headache, phantom limb pain, peripheral nerve injury, and combinations thereof.

Another embodiment of this embodiment is wherein the disease or condition is selected from the group consisting of pain associated with HIV, HIV treatment induced neuropathy, trigeminal neuralgia, post-herpetic neuralgia, eudynia, heat sensitivity, tosarcoidosis, irritable bowel syndrome, Crohns disease, pain associated with multiple sclerosis (MS), amyotrophic lateral sclerosis (ALS), diabetic neuropathy, peripheral neuropathy, arthritic, rheumatoid arthritis, osteoarthritis, atherosclerosis, paroxysmal dystonia, myasthenia syndromes, myotonia, malignant hyperthermia, cystic fibrosis, pseudoaldosteronism, rhabdomyolysis, hypothyroidism, bipolar depression, anxiety, schizophrenia, sodium channel toxin related illnesses, familial erythermalgia, primary erythermalgia, familial rectal pain, cancer, epilepsy, partial and general tonic seizures, restless leg syndrome, arrhythmias, fibromyalgia, neuroprotection under ischaemic conditions caused by stroke or neural trauma, tachy-arrhythmias, atrial fibrillation and ventricular fibrillation.

Another embodiment of the invention is the method of treating pain in a mammal, preferably a human, by the inhibition of ion flux through a voltage-dependent sodium channel in the mammal, wherein the method comprises administering to the mammal in need thereof a therapeutically effective amount of an embodiment of a compound of the invention, as set forth above, as a stereoisomer, enantiomer, tautomer thereof or mixtures thereof, or a pharmaceutically acceptable salt, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

Another embodiment of the invention is the method of treating or preventing hypercholesterolemia in a mammal, preferably a human, wherein the method comprises administering to the mammal in need thereof a therapeutically effective amount of an embodiment of a compound of the invention, as set forth above, as a stereoisomer, enantiomer, tautomer thereof or mixtures thereof, or a pharmaceutically acceptable salt, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

Another embodiment of the invention is the method of treating or preventing benign prostatic hyperplasia in a mammal, preferably a human, wherein the method comprises administering to the mammal in need thereof a therapeutically effective amount of an embodiment of a compound of the invention, as set forth above, as a stereoisomer, enantiomer, tautomer thereof or mixtures thereof, or a pharmaceutically acceptable salt, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

Another embodiment of the invention is the method of treating or preventing pruritis in a mammal, preferably a human, wherein the method comprises administering to the mammal in need thereof a therapeutically effective amount of an embodiment of a compound of the invention, as set forth above, as a stereoisomer, enantiomer, tautomer thereof or mixtures thereof, or a pharmaceutically acceptable salt, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

Another embodiment of the invention is the method of treating or preventing cancer in a mammal, preferably a human, wherein the method comprises administering to the mammal in need thereof a therapeutically effective amount of an embodiment of a compound of the invention, as set forth above, as a stereoisomer, enantiomer, tautomer thereof or mixtures thereof, or a pharmaceutically acceptable salt, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

Another embodiment of the invention is the method of decreasing ion flux through a voltage-dependent sodium channel in a cell in a mammal, wherein the method comprises contacting the cell with an embodiment of a compound of the invention, as set forth above, as a stereoisomer, enantiomer, tautomer thereof or mixtures thereof, or a pharmaceutically acceptable salt, solvate or prodrug thereof.

Specific embodiments of the compounds of the invention are described in more detail below in the Preparation of the Compounds of the Invention.

### Utility and Testing of the Compounds of the Invention

The compounds of the invention modulate, preferably inhibit, ion flux through a voltage-dependent sodium channel in a mammal, especially in a human. Any such modulation, whether it be partial or complete inhibition or prevention of ion flux, is sometimes referred to herein as “blocking” and corresponding compounds as “blockers” or “inhibitors”. In general, the compounds of the invention modulate the activity of a sodium channel downwards, inhibit the voltage-dependent activity of the sodium channel, and/or reduce or prevent sodium ion flux across a cell membrane by preventing sodium channel activity such as ion flux.

The compounds of the invention inhibit the ion flux through a voltage-dependent sodium channel. Preferably, the compounds are state or frequency dependent modifiers of the sodium channels, having a low affinity for the rested/closed state and a high affinity for the inactivated state. These compounds are likely to interact with overlapping sites located in the inner cavity of the sodium conducting pore of the channel similar to that described for other state-dependent sodium channel blockers (Cestèle, S., et al., op. cit.). These compounds may also be likely to interact with sites outside of the inner cavity and have allosteric effects on sodium ion conduction through the channel pore.

Any of these consequences may ultimately be responsible for the overall therapeutic benefit provided by these compounds.

Accordingly, the compounds of the invention are sodium channel blockers and are therefore useful for treating diseases and conditions in mammals, preferably humans, and other organisms, including all those human diseases and conditions which are the result of aberrant voltage-dependent sodium channel biological activity or which may be ameliorated by modulation of voltage-dependent sodium channel biological activity.

As defined herein, a sodium channel-mediated disease or condition refers to a disease or condition in a mammal, preferably a human, which is ameliorated upon modulation of the sodium channel and includes, but is not limited to, pain, central nervous conditions such as epilepsy, anxiety, depression and bipolar disease; cardiovascular conditions such as arrhythmias, atrial fibrillation and ventricular fibrillation; neuromuscular conditions such as restless leg syndrome and muscle paralysis or tetanus; neuroprotection against stroke, neural trauma and multiple sclerosis; and channelopathies such as erythromyalgia and familial rectal pain syndrome.

The present invention therefore relates to compounds, pharmaceutical compositions and methods of using the compounds and pharmaceutical compositions for the treatment of sodium channel-mediated diseases in mammals, preferably humans and preferably diseases related to pain, central nervous conditions such as epilepsy, anxiety, depression and bipolar disease; cardiovascular conditions such as arrhythmias, atrial fibrillation and ventricular fibrillation; neuromuscular conditions such as restless leg syndrome and muscle paralysis or tetanus; neuroprotection against stroke, neural trauma and multiple sclerosis; and channelopathies such as erythromyalgia and familial rectal pain syndrome, by administering to a mammal, preferably a human, in need of such treatment an effective amount of a sodium channel blocker modulating, especially inhibiting, agent.

Accordingly, the present invention provides a method for treating a mammal for, or protecting a mammal from developing, a sodium channel-mediated disease, especially pain, comprising administering to the mammal, especially a human, in need thereof, a therapeutically effective amount of a compound of the invention or a pharmaceutical composition comprising a therapeutically effective amount of a compound of the invention wherein the compound modulates the activity of one or more voltage-dependent sodium channels.

The general value of the compounds of the invention in mediating, especially inhibiting, the sodium channel ion flux can be determined using the assays described below in the Biological Assays section. Alternatively, the general value of the compounds in treating conditions and diseases in humans may be established in industry standard animal models for demonstrating the efficacy of compounds in treating pain. Animal models of human neuropathic pain conditions have been developed that result in reproducible sensory deficits (allodynia, hyperalgesia, and spontaneous pain) over a sustained period of time that can be evaluated by sensory testing. By establishing the degree of mechanical, chemical, and temperature induced allodynia and hyperalgesia present, several physiopathological conditions observed in humans can be modeled allowing the evaluation of pharmacotherapies.

In rat models of peripheral nerve injury, ectopic activity in the injured nerve corresponds to the behavioural signs of pain. In these models, intravenous application of the sodium channel blocker and local anesthetic lidocaine can suppress the ectopic activity and reverse the tactile allodynia at concentrations that do not affect general behaviour and motor function (Mao, J. and Chen, L. L, Pain (2000), 87:7-17). Allimetric scaling of the doses effective in these rat models, translates into doses similar to those shown to be efficacious in humans (Tanelian, D. L. and Brose, W. G., Anesthesiology (1991), 74(5):949-951). Furthermore, Lidoderm®, lidocaine applied in the form of a dermal patch, is currently an FDA approved treatment for post-herpetic neuralgia (Devers, A. and Glaler, B. S., Clin. J. Pain (2000), 16(3):205-8).

A sodium channel-mediated disease or condition also includes pain associated with HIV, HIV treatment induced neuropathy, trigeminal neuralgia, glossopharyngeal neuralgia, neuropathy secondary to metastatic infiltration, adiposis dolorosa, thalamic lesions, hypertension, autoimmune disease, asthma, drug addiction (e.g. opiate, benzodiazepine, amphetamine, cocaine, alcohol, butane inhalation), Alzheimer, dementia, age-related memory impairment, Korsakoff syndrome, restenosis, urinary dysfunction, incontinence, Parkinson's disease, cerebrovascular ischemia, neurosis, gastrointestinal disease, sickle cell anemia, transplant rejection, heart failure, myocardial infarction, reperfusion injury, intermittant claudication, angina, convulsion, respiratory disorders, cerebral or myocardial ischemias, long-QT syndrome, Catecholeminergic polymorphic ventricular tachycardia, ophthalmic diseases, spasticity, spastic paraplegia, myopathies, myasthenia gravis, paramyotonia congentia, hyperkalemic periodic paralysis, hypokalemic periodic paralysis, alopecia, anxiety disorders, psychotic disorders, mania, paranoia, seasonal affective disorder, panic disorder, obsessive compulsive disorder (OCD), phobias, autism, Aspergers Syndrome, Retts syndrome, disintegrative disorder, attention deficit disorder, aggressivity, impulse control disorders, thrombosis, pre clampsia, congestive cardiac failure, cardiac arrest, Freidrich's ataxia, Spinocerebellear ataxia, myelopathy, radiculopathy, systemic lupus erythamatosis, granulomatous disease, olivo-ponto-cerebellar atrophy, spinocerebellar ataxia, episodic ataxia, myokymia, progressive pallidal atrophy, progressive supranuclear palsy and spasticity, traumatic brain injury, cerebral oedema, hydrocephalus injury, spinal cord injury, anorexia nervosa, bulimia, Prader-Willi syndrome, obesity, optic neuritis, cataract, retinal haemorrhage, ischaemic retinopathy, retinitis pigmentosa, acute and chronic glaucoma, macular degeneration, retinal artery occlusion, Chorea, Huntington's chorea, cerebral edema, proctitis, post-herpetic neuralgia, eudynia, heat sensitivity, sarcoidosis, irritable bowel syndrome, Tourette syndrome, Lesch-Nyhan Syndrome, Brugado syndrome, Liddle syndrome, Crohns disease, multiple sclerosis and the pain associated with multiple sclerosis (MS), amyotrophic lateral sclerosis (ALS), disseminated sclerosis, diabetic neuropathy, peripheral neuropathy, charcot marie tooth syndrome, arthritic, rheumatoid arthritis, osteoarthritis, chondrocalcinosis, atherosclerosis, paroxysmal dystonia, myasthenia syndromes, myotonia, myotonic dystrophy, muscular dystrophy, malignant hyperthermia, cystic fibrosis, pseudoaldosteronism, rhabdomyolysis, mental handicap, hypothyroidism, bipolar depression, anxiety, schizophrenia, sodium channel toxin related illnesses, familial erythermalgia, primary erythermalgia, rectal pain, cancer, epilepsy, partial and general tonic seizures, febrile seizures, absence seizures (petit mal), myoclonic seizures, atonic seizures, clonic seizures, Lennox Gastaut, West Syndrome (infantile spasms), multiresistant seizures, seizure prophylaxis (anti-epileptogenic), familial Mediterranean fever syndrome, gout, restless leg syndrome, arrhythmias, fibromyalgia, neuroprotection under ischaemic conditions caused by stroke or neural trauma, tachy-arrhythmias, atrial fibrillation and ventricular fibrillation and as a general or local anaesthetic.

As used herein, the term “pain” refers to all categories of pain and is recognized to include, but is not limited to, neuropathic pain, inflammatory pain, nociceptive pain, idiopathic pain, neuralgic pain, orofacial pain, burn pain, burning mouth syndrome, somatic pain, visceral pain, myofacial pain, dental pain, cancer pain, chemotherapy pain, trauma pain, surgical pain, post-surgical pain, childbirth pain, labor pain, reflex sympathetic dystrophy, brachial plexus avulsion, neurogenic bladder, acute pain (e.g. musculoskeletal and post-operative pain), chronic pain, persistent pain, peripherally mediated pain, centrally mediated pain, chronic headache, migraine headache, familial hemiplegic migraine, conditions associated with cephalic pain, sinus headache, tension headache, phantom limb pain, peripheral nerve injury, pain following stroke, thalamic lesions, radiculopathy, HIV pain, post-herpetic pain, non-cardiac chest pain, irritable bowel syndrome and pain associated with bowel disorders and dyspepsia, and combinations thereof.

Sodium channel blockers have clinical uses in addition to pain. Epilepsy and cardiac arrhythmias are often targets of sodium channel blockers. Recent evidence from animal models suggest that sodium channel blockers may also be useful for neuroprotection under ischaemic conditions caused by stroke or neural trauma and in patients with multiple sclerosis (MS) (Clare, J. J. et al., op. cit. and Anger, T. et al., op. cit.).

The present invention also relates to compounds, pharmaceutical compositions and methods of using the compounds and pharmaceutical compositions for the treatment or prevention of diseases or conditions such as benign prostatic hyperplasia (BPH), hypercholesterolemia, cancer and pruritis (itch).

Benign prostatic hyperplasia (BPH), also known as benign prostatic hypertrophy, is one of the most common diseases affecting aging men. BPH is a progressive condition which is characterized by a nodular enlargement of prostatic tissue resulting in obstruction of the urethra. Consequences of BPH can include hypertrophy of bladder smooth muscle, a decompensated bladder, acute urinary retention and an increased incidence of urinary tract infection.

BPH has a high public health impact and is one of the most common reasons for surgical intervention among elderly men. Attempts have been made to clarify the etiology and pathogenesis and, to that end, experimental models have been developed. Spontaneous animal models are limited to the chimpanzee and the dog. BPH in man and the dog share many common features. In both species, the development of BPH occurs spontaneously with advanced age and can be prevented by early/prepubertal castration. A medical alternative to surgery is very desirable for treating BHP and the consequences.

The prostatic epithelial hyperplasia in both man and the dog is androgen sensitive, undergoing involution with androgen deprivation and resuming epithelial hyperplasia when androgen is replaced. Cells originating from the prostate gland have been shown to express high levels of voltage gated sodium channels. Immunostaining studies clearly demonstrated evidence for voltage gated sodium channels in prostatic tissues (Prostate Cancer Prostatic Dis. 2005; 8(3):266-73).

Hypercholesterolemia, i.e., elevated blood cholesterol, is an established risk factor in the development of, e.g., atherosclerosis, coronary artery disease, hyperlipidemia, stroke, hyperinsulinemias, hypertension, obesity, diabetes, cardiovascular diseases (CVD), myocardial ischemia, and heart attack. Thus, lowering the levels of total serum cholesterol in individuals with high levels of cholesterol has been known to reduce the risk of these diseases. The lowering of low density lipoprotein cholesterol in particular is an essential step in the prevention of CVD. Although there are a variety of hypercholesterolemia therapies, there is a continuing need and a continuing search in this field of art for alternative therapies.

The invention provides compounds which are useful as antihypercholesterolemia agents and their related conditions. The present compounds may act in a variety of ways. While not wishing to be bound to any particular mechanism of action, the compounds may be direct or indirect inhibitors of the enzyme acyl CoA: cholesterol acyl transferase (ACAT) that results in inhibition of the esterification and transport of cholesterol across the intestinal wall. Another possibility may be that the compounds of the invention may be direct or indirect inhibitors of cholesterol biosynthesis in the liver. It is possible that some compounds of the invention may act as both direct or indirect inhibitors of ACAT and cholesterol biosynthesis.

Pruritus, commonly known as itch, is a common dermatological condition. While the exact causes of pruritis are complex and poorly understood, there has long been acknowledged to have interactions with pain. In particular, it is believed that sodium channels likely communicate or propagate along the nerve axon the itch signals along the skin. Transmission of the itch impulses results in the unpleasant sensation that elicits the desire or reflex to scratch.

From a neurobiology level, it is believed that there is a shared complexity of specific mediators, related neuronal pathways and the central processes of itch and pain and recent data suggest that there is a broad overlap between pain- and itch-related peripheral mediators and/or receptors (Ikoma et al., Nature Reviews Neuroscience, 7:535-547, 2006). Remarkably, pain and itch have similar mechanisms of neuronal sensitization in the peripheral nervous system and the central nervous system but exhibits intriguing differences as well.

For example, the mildly painful stimuli from scratching are effective in abolishing the itch sensation. In contrast, analgesics such as opioids can generate severe pruritus. The antagonistic interaction between pain and itch can be exploited in pruritus therapy, and current research concentrates on the identification of common targets for future analgesic and antipruritic therapy.

Compounds of the present invention have been shown to have analgesic effects in a number of animal models at oral doses ranging from 1 mg/Kg to 100 mg/Kg. The compounds of the invention can also be useful for treating pruritus.

The types of itch or skin irritation, include, but are not limited to:

a) psoriatic pruritis, itch due to hemodyalisis, aguagenic pruritus, and itching caused by skin disorders (e.g., contact dermatitis), systemic disorders, neuropathy, psychogenic factors or a mixture thereof;

b) itch caused by allergic reactions, insect bites, hypersensitivity (e.g., dry skin, acne, eczema, psoriasis), inflammatory conditions or injury;

c) itch associated with vulvar vestibulitis; and

d) skin irritation or inflammatory effect from administration of another therapeutic such as, for example, antibiotics, antivirals and antihistamines.

The compounds of the invention are also useful in treating or preventing certain hormone sensitive cancers, such as prostate cancer (adenocarcinoma), breast cancer, ovarian cancer, testicular cancer, thyroid neoplasia, in a mammal, preferably a human. The voltage gated sodium channels have been demonstrated to be expressed in prostate and breast cancer cells. Up-regulation of neonatal Naᵥ1.5 occurs as an integral part of the metastatic process in human breast cancer and could serve both as a novel marker of the metastatic phenotype and a therapeutic target (Clin. Cancer Res. 2005, Aug. 1; 11(15): 5381-9). Functional expression of voltage-gated sodium channel alpha-subunits, specifically Naᵥ1.7, is associated with strong metastatic potential in prostate cancer (CaP) in vitro. Voltage-gated sodium channel alpha-subunits immunostaining, using antibodies specific to the sodium channel alpha subunit was evident in prostatic tissues and markedly stronger in CaP vs non-CaP patients (Prostate Cancer Prostatic Dis., 2005; 8(3):266-73).

The compounds of the invention are also useful in treating or preventing symptoms in a mammal associated with BPH such as, but not limited to, acute urinary retention and urinary tract infection.

The compounds of the invention are also useful in treating or preventing certain endocrine imbalances or endocrinopathies such as congenital adrenal hyperplasia , hyperthyroidism, hypothyroidism, osteoporosis, osteomalacia, rickets, Cushing's Syndrome, Conn's syndrome, hyperaldosteronism, hypogonadism, hypergonadism, infertility, fertility and diabetes.

The present invention readily affords many different means for identification of sodium channel modulating agents that are useful as therapeutic agents. Identification of modulators of sodium channel can be assessed using a variety of in vitro and in vivo assays, e.g. measuring current, measuring membrane potential, measuring ion flux, (e.g. sodium or guanidinium), measuring sodium concentration, measuring second messengers and transcription levels, and using e.g., voltage-sensitive dyes, radioactive tracers, and patch-clamp electrophysiology.

One such protocol involves the screening of chemical agents for ability to modulate the activity of a sodium channel thereby identifying it as a modulating agent.

A typical assay described in Bean et al., J. General Physiology (1983), 83:613-642, and Leuwer, M., et al., Br. J. Pharmacol (2004), 141(1):47-54, uses patch-clamp techniques to study the behaviour of channels. Such techniques are known to those skilled in the art, and may be developed, using current technologies, into low or medium throughput assays for evaluating compounds for their ability to modulate sodium channel behaviour.

A competitive binding assay with known sodium channel toxins such as tetrodotoxin, alpha-scorpion toxins, aconitine, BTX and the like, may be suitable for identifying potential therapeutic agents with high selectivity for a particular sodium channel. The use of BTX in such a binding assay is well known and is described in McNeal, E. T., et al., J. Med. Chem. (1985), 28(3):381-8; and Creveling, C. R., et al., Methods in Neuroscience, Vol. 8: Neurotoxins (Conn PM Ed) (1992), pp. 25-37, Academic Press, New York.

These assays can be carried out in cells, or cell or tissue extracts expressing the channel of interest in a natural endogenous setting or in a recombinant setting. The assays that can be used include plate assays which measure Na+ influx through surrogate markers such as ¹⁴C-guanidine influx or determine cell depolarization using fluorescent dyes such as the FRET based and other fluorescent assays or a radiolabelled binding assay employing radiolabelled aconitine, BTX, TTX or STX. More direct measurements can be made with manual or automated electrophysiology systems. The guanidine influx assay is explained in more detail below in the Biological Assays section.

Throughput of test compounds is an important consideration in the choice of screening assay to be used. In some strategies, where hundreds of thousands of compounds are to be tested, it is not desirable to use low throughput means. In other cases, however, low throughput is satisfactory to identify important differences between a limited number of compounds. Often it will be necessary to combine assay types to identify specific sodium channel modulating compounds.

Electrophysiological assays using patch clamp techniques is accepted as a gold standard for detailed characterization of sodium channel compound interactions, and as described in Bean et al., op. cit. and Leuwer, M., et al., op. cit. There is a manual low-throughput screening (LTS) method which can compare 2-10 compounds per day; a recently developed system for automated medium-throughput screening (MTS) at 20-50 patches (i.e. compounds) per day; and a technology from Molecular Devices Corporation (Sunnyvale, Calif.) which permits automated high-throughput screening (HTS) at 1000-3000 patches (i.e. compounds) per day.

One automated patch-clamp system utilizes planar electrode technology to accelerate the rate of drug discovery. Planar electrodes are capable of achieving high-resistance, cells-attached seals followed by stable, low-noise whole-cell recordings that are comparable to conventional recordings. A suitable instrument is the PatchXpress 7000A (Axon Instruments Inc, Union City, Calif.). A variety of cell lines and culture techniques, which include adherent cells as well as cells growing spontaneously in suspension are ranked for seal success rate and stability. Immortalized cells (e.g. HEK and CHO) stably expressing high levels of the relevant sodium ion channel can be adapted into high-density suspension cultures.

Other assays can be selected which allow the investigator to identify compounds which block specific states of the channel, such as the open state, closed state or the resting state, or which block transition from open to closed, closed to resting or resting to open. Those skilled in the art are generally familiar with such assays.

Binding assays are also available, however these are of only limited functional value and information content. Designs include traditional radioactive filter based binding assays or the confocal based fluorescent system available from Evotec OAI group of companies (Hamburg, Germany), both of which are HTS.

Radioactive flux assays can also be used. In this assay, channels are stimulated to open with veratridine or aconitine and held in a stabilized open state with a toxin, and channel blockers are identified by their ability to prevent ion influx. The assay can use radioactive ²²[Na] and ¹⁴[C] guanidinium ions as tracers. FlashPlate & Cytostar-T plates in living cells avoids separation steps and are suitable for HTS. Scintillation plate technology has also advanced this method to HTS suitability. Because of the functional aspects of the assay, the information content is reasonably good.

Yet another format measures the redistribution of membrane potential using the FLIPR system membrane potential kit (HTS) available from Molecular Dynamics (a division of Amersham Biosciences, Piscataway, N.J.). This method is limited to slow membrane potential changes. Some problems may result from the fluorescent background of compounds. Test compounds may also directly influence the fluidity of the cell membrane and lead to an increase in intracellular dye concentrations. Still, because of the functional aspects of the assay, the information content is reasonably good.

Sodium dyes can be used to measure the rate or amount of sodium ion influx through a channel. This type of assay provides a very high information content regarding potential channel blockers. The assay is functional and would measure Na+ influx directly. CoroNa Red, SBFI and/or sodium green (Molecular Probes, Inc. Eugene Oreg.) can be used to measure Na influx; all are Na responsive dyes. They can be used in combination with the FLIPR instrument. The use of these dyes in a screen has not been previously described in the literature. Calcium dyes may also have potential in this format.

In another assay, FRET based voltage sensors are used to measure the ability of a test compound to directly block Na influx. Commercially available HTS systems include the VIPR™ II FRET system (Aurora Biosciences Corporation, San Diego, Calif., a division of Vertex Pharmaceuticals, Inc.) which may be used in conjunction with FRET dyes, also available from Aurora Biosciences. This assay measures sub-second responses to voltage changes. There is no requirement for a modifier of channel function. The assay measures depolarization and hyperpolarizations, and provides ratiometric outputs for quantification. A somewhat less expensive MTS version of this assay employs the FLEXstation™ (Molecular Devices Corporation) in conjunction with FRET dyes from Aurora Biosciences. Other methods of testing the compounds disclosed herein are also readily known and available to those skilled in the art.

These results provide the basis for analysis of the structure-activity relationship (SAR) between test compounds and the sodium channel. Certain substituents on the core structure of the test compound tend to provide more potent inhibitory compounds. SAR analysis is one of the tools those skilled in the art may now employ to identify preferred embodiments of the compounds of the invention for use as therapeutic agents.

Modulating agents so identified are then tested in a variety of in vivo models so as to determine if they alleviate pain, especially chronic pain or other conditions such as arrhythmias and epilepsy, benign prostatic hyperplasia (BPH), hypercholesterolemia, cancer and pruritis (itch) with minimal adverse events. The assays described below in the Biological Assays Section are useful in assessing the biological activity of the instant compounds.

Typically, a successful therapeutic agent of the present invention will meet some or all of the following criteria. Oral availability should be at or above 20%. Animal model efficacy is less than about 0.1 μg to about 100 mg/Kg body weight and the target human dose is between 0.1 μg to about 100 mg/Kg body weight, although doses outside of this range may be acceptable (“mg/Kg” means milligrams of compound per kilogram of body mass of the subject to whom it is being administered). The therapeutic index (or ratio of toxic dose to therapeutic dose) should be greater than 100. The potency (as expressed by IC₅₀ value) should be less than 100 μM, preferably below 10 μM, more preferably below 1 μM and most preferably below 50 nM. The IC₅₀ (“Inhibitory Concentration—50%”) is a measure of the amount of compound required to achieve 50% inhibition of ion flux through a sodium channel, over a specific time period, in an assay of the invention. Compounds of the present invention in the guanidine influx assay have demonstrated IC₅₀'s ranging from less than a nanomolar to less than 100 micromolar.

In an alternative use of the invention, the compounds of the invention can be used in in vitro or in vivo studies as exemplary agents for comparative purposes to find other compounds also useful in treatment of, or protection from, the various diseases disclosed herein.

Another aspect of the invention relates to inhibiting Naᵥ1.1, Naᵥ1.2, Naᵥ1.3, Naᵥ1.4, Naᵥ1.5, Naᵥ1.6, Naᵥ1.7, Naᵥ1.8, or Naᵥ1.9 activity in a biological sample or a mammal, preferably a human, which method comprises administering to the mammal, preferably a human, or contacting said biological sample with a compound of formula I or a composition comprising said compound. The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of Naᵥ1.1, Naᵥ1.2, Naᵥ1.3, Naᵥ1.4, Naᵥ1.5, Naᵥ1.6, Naᵥ1.7, Naᵥ1.8, or Naᵥ1.9 activity in a biological sample is useful for a variety of purposes that are known to one of skill in the art. Examples of such purposes include, but are not limited to, the study of sodium ion channels in biological and pathological phenomena; and the comparative evaluation of new sodium ion channel inhibitors.

The compounds of the invention, as set forth above in the Summary of the Invention, as stereoisomers, enantiomers, tautomers thereof or mixtures thereof, or pharmaceutically acceptable salts, solvates or prodrugs thereof, and/or the pharmaceutical compositions described herein which comprise a pharmaceutically acceptable excipient and one or more compounds of the invention, as set forth above in the Summary of the Invention, as a stereoisomer, enantiomer, tautomer thereof or mixtures thereof, or a pharmaceutically acceptable salt, solvate or prodrug thereof, can be used in the preparation of a medicament for the treatment of sodium channel-mediated disease or condition in a mammal.

### Pharmaceutical Compositions of the Invention and Administration

The present invention also relates to pharmaceutical composition containing the compounds of the invention disclosed herein. In one embodiment, the present invention relates to a composition comprising compounds of the invention in a pharmaceutically acceptable carrier, excipient or diluent and in an amount effective to modulate, preferably inhibit, ion flux through a voltage-dependent sodium channel to treat sodium channel mediated diseases, such as pain, when administered to an animal, preferably a mammal, most preferably a human patient.

Administration of the compounds of the invention, or their pharmaceutically acceptable salts, in pure form or in an appropriate pharmaceutical composition, can be carried out via any of the accepted modes of administration of agents for serving similar utilities. The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see The Science and Practice of Pharmacy, 20th Edition (Philadelphia College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings of this invention.

The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids, such as water, saline, glycerol and ethanol, and the like. A thorough discussion of pharmaceutically acceptable carriers, diluents, and other excipients is presented in REMINGTON'S PHARMACEUTICAL SCIENCES (Mack Pub. Co., N.J. current edition).

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the invention intended for either parenteral or oral administration should contain an amount of a compound of the invention such that a suitable dosage will be obtained. Typically, this amount is at least 0.01% of a compound of the invention in the composition. When intended for oral administration, this amount may be varied to be between 0.1 and about 70% of the weight of the composition. Preferred oral pharmaceutical compositions contain between about 4% and about 50% of the compound of the invention. Preferred pharmaceutical compositions and preparations according to the present invention are prepared so that a parenteral dosage unit contains between 0.01 to 10% by weight of the compound prior to dilution of the invention.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the invention from about 0.1 to about 10% w/v (weight per unit volume).

The pharmaceutical composition of the invention may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the invention may be prepared by methodology well known in the pharmaceutical art. For example, a pharmaceutical composition intended to be administered by injection can be prepared by combining a compound of the invention with sterile, distilled water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The compounds of the invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 Kg mammal) from about 0.001 mg/Kg (i.e., 0.07 mg) to about 100 mg/Kg (i.e., 7.0 g); preferably a therapeutically effective dose is (for a 70 Kg mammal) from about 0.01 mg/Kg (i.e., 0.7 mg) to about 50 mg/Kg (i.e., 3.5 g); more preferably a therapeutically effective dose is (for a 70 Kg mammal) from about 1 mg/Kg (i.e., 70 mg) to about 25 mg/Kg (i.e., 1.75 g).

The ranges of effective doses provided herein are not intended to be limiting and represent preferred dose ranges. However, the most preferred dosage will be tailored to the individual subject, as is understood and determinable by one skilled in the relevant arts. (see, e.g., Berkow et al., eds., The Merck Manual, 16^{th} edition, Merck and Co., Rahway, N.J., 1992; Goodmanetna., eds., Goodman and Cilman's The Pharmacological Basis of Therapeutics, 10^{th} edition, Pergamon Press, Inc., Elmsford, N.Y., (2001); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985); Osolci al., eds., Remington's Pharmaceutical Sciences, 18^{th} edition, Mack Publishing Co., Easton, Pa. (1990); Katzung, Basic and Clinical Pharmacology, Appleton and Lange, Norwalk, Conn. (1992)).

The total dose required for each treatment can be administered by multiple doses or in a single dose over the course of the day, if desired. Generally, treatment is initiated with smaller dosages, which are less than the optimum dose of the compound. Thereafter, the dosage is increased by small increments until the optimum effect under the circumstances is reached. The diagnostic pharmaceutical compound or composition can be administered alone or in conjunction with other diagnostics and/or pharmaceuticals directed to the pathology, or directed to other symptoms of the pathology. The recipients of administration of compounds and/or compositions of the invention can be any vertebrate animal, such as mammals. Among mammals, the preferred recipients are mammals of the Orders Primate (including humans, apes and monkeys), Arteriodactyla (including horses, goats, cows, sheep, pigs), Rodenta (including mice, rats, rabbits, and hamsters), and Carnivora (including cats, and dogs). Among birds, the preferred recipients are turkeys, chickens and other members of the same order. The most preferred recipients are humans.

For topical applications, it is preferred to administer an effective amount of a pharmaceutical composition according to the invention to target area, e.g., skin surfaces, mucous membranes, and the like, which are adjacent to peripheral neurons which are to be treated. This amount will generally range from about 0.0001 mg to about 1 g of a compound of the invention per application, depending upon the area to be treated, whether the use is diagnostic, prophylactic or therapeutic, the severity of the symptoms, and the nature of the topical vehicle employed. A preferred topical preparation is an ointment, wherein about 0.001 to about 50 mg of active ingredient is used per cc of ointment base. The pharmaceutical composition can be formulated as transdermal compositions or transdermal delivery devices (“patches”). Such compositions include, for example, a backing, active compound reservoir, a control membrane, liner and contact adhesive. Such transdermal patches may be used to provide continuous pulsatile, or on demand delivery of the compounds of the present invention as desired.

The compositions of the invention can be formulated so as to provide quick, sustained or delayed release of the active ingredient after administration to the patient by employing procedures known in the art. Controlled release drug delivery systems include osmotic pump systems and dissolutional systems containing polymer-coated reservoirs or drug-polymer matrix formulations. Examples of controlled release systems are given in U.S. Pat. Nos. 3,845,770 and 4,326,525 and in P. J. Kuzma et al., Regional Anesthesia (1997), 22(6):543-551, all of which are incorporated herein by reference.

The compositions of the invention can also be delivered through intra-nasal drug delivery systems for local, systemic, and nose-to-brain medical therapies. Controlled Particle Dispersion (CPD)™ technology, traditional nasal spray bottles, inhalers or nebulizers are known by those skilled in the art to provide effective local and systemic delivery of drugs by targeting the olfactory region and paranasal sinuses.

The invention also relates to an intravaginal shell or core drug delivery device suitable for administration to the human or animal female. The device may be comprised of the active pharmaceutical ingredient in a polymer matrix, surrounded by a sheath, and capable of releasing the compound in a substantially zero order pattern on a daily basis similar to devises used to apply testosterone as described in PCT Published Patent Application No. WO 98/50016.

Current methods for ocular delivery include topical administration (eye drops), subconjunctival injections, periocular injections, intravitreal injections, surgical implants and iontophoresis (uses a small electrical current to transport ionized drugs into and through body tissues). Those skilled in the art would combine the best suited excipients with the compound for safe and effective intra-occular administration.

The most suitable route will depend on the nature and severity of the condition being treated. Those skilled in the art are also familiar with determining administration methods (e.g., oral, intravenous, inhalation, sub-cutaneous, rectal etc.), dosage forms, suitable pharmaceutical excipients and other matters relevant to the delivery of the compounds to a subject in need thereof.

### Combination Therapy

The compounds of the invention may be usefully combined with one or more other compounds of the invention or one or more other therapeutic agent or as any combination thereof, in the treatment of sodium channel-mediated diseases and conditions. For example, a compound of the invention may be administered simultaneously, sequentially or separately in combination with other therapeutic agents, including, but not limited to:
opiates analgesics, e.g. morphine, heroin, cocaine, oxymorphine, levorphanol, levallorphan, oxycodone, codeine, dihydrocodeine, propoxyphene, nalmefene, fentanyl, hydrocodone, hydromorphone, meripidine, methadone, nalorphine, naloxone, naltrexone, buprenorphine, butorphanol, nalbuphine and pentazocine;
non-opiate analgesics, e.g. acetomeniphen, salicylates (e.g. aspirin);
nonsteroidal antiinflammatory drugs (NSAIDs), e.g. ibuprofen, naproxen, fenoprofen, ketoprofen, celecoxib, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin and zomepirac;
anticonvulsants, e.g. carbamazepine, oxcarbazepine, lamotrigine, valproate, topiramate, gabapentin and pregabalin;
antidepressants such as tricyclic antidepressants, e.g. amitriptyline, clomipramine, despramine, imipramine and nortriptyline;
COX-2 selective inhibitors, e.g. celecoxib, rofecoxib, parecoxib, valdecoxib, deracoxib, etoricoxib, and lumiracoxib;
alpha-adrenergics, e.g. doxazosin, tamsulosin, clonidine, guanfacine, dexmetatomidine, modafinil, and 4-amino-6,7-dimethoxy-2-(5-methane sulfonamido-1,2,3,4-tetrahydroisoquinol-2-yl)-5-(2-pyridyl)quinazoline;
barbiturate sedatives, e.g. amobarbital, aprobarbital, butabarbital, butabital, mephobarbital, metharbital, methohexital, pentobarbital, phenobartital, secobarbital, talbutal, theamylal and thiopental;
tachykinin (NK) antagonist, particularly an NK-3, NK-2 or NK-1 antagonist, e.g. (αR,9R)-7-[3,5-bis(trifluoromethyl)benzyl)]-8,9,10,11-tetrahydro-9-methyl-5-(4-methylphenyl)-7H-[1,4]diazocino[2,1-g][1,7]-naphthyridine-6-13-dione (TAK-637), 5-[[2R,3S)-2-[(1R)-1-[3,5-bis(trifluoromethylphenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy5-(trifluoromethoxy)phenyl]-methylamino]-2-phenylpiperidine (2S,3S);
coal-tar analgesics, in particular paracetamol;
serotonin reuptake inhibitors, e.g. paroxetine, sertraline, norfluoxetine (fluoxetine desmethyl metabolite), metabolite demethylsertraline, ‘3 fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine, trazodone and fluoxetine;
noradrenaline (norepinephrine) reuptake inhibitors, e.g. maprotiline, lofepramine, mirtazepine, oxaprotiline, fezolamine, tomoxetine, mianserin, buproprion, buproprion metabolite hydroxybuproprion, nomifensine and viloxazine (Vivalan®)), especially a selective noradrenaline reuptake inhibitor such as reboxetine, in particular (S,S)-reboxetine, and venlafaxine duloxetine neuroleptics sedative/anxiolytics;
dual serotonin-noradrenaline reuptake inhibitors, such as venlafaxine, venlafaxine metabolite O-desmethylvenlafaxine, clomipramine, clomipramine metabolite desmethylclomipramine, duloxetine, milnacipran and imipramine;
acetylcholinesterase inhibitors such as donepezil;
metabotropic glutamate receptor (mGluR) antagonists;
local anaesthetic such as mexiletine and lidocaine;
corticosteroid such as dexamethasone;
antiarrhythimics, e.g., mexiletine and phenytoin;
muscarinic antagonists, e.g., tolterodine, propiverine, tropsium t chloride, darifenacin, solifenacin, temiverine and ipratropium;
cannabinoids;
vanilloid receptor agonists (e.g. resinferatoxin) or antagonists (e.g. capsazepine);
sedatives, e.g. glutethimide, meprobamate, methaqualone, and dichloralphenazone;
anxiolytics such as benzodiazepines,
antidepressants such as mirtazapine,
topical agents (e.g. lidocaine, capsacin and resiniferotoxin);
muscle relaxants such as benzodiazepines, baclofen, carisoprodol, chlorzoxazone, cyclobenzaprine, methocarbamol and orphrenadine;
anti-histamines or H1 antagonists;
NMDA receptor antagonists;
5-HT receptor agonists/antagonists;
PDEV inhibitors;
Tramadol®;
cholinergic (nicotinc) analgesics;
alpha-2-delta ligands;
prostaglandin E2 subtype antagonists;
leukotriene B4 antagonists;
5-lipoxygenase inhibitors.

Sodium channel-mediated diseases and conditions that may be treated and/or prevented using such combinations include but not limited to, pain, central and peripherally mediated, acute, chronic, neuropathic as well as other diseases with associated pain and other central nervous disorders such as epilepsy, anxiety, depression and bipolar disease; or cardiovascular disorders such as arrhythmias, atrial fibrillation and ventricular fibrillation; neuromuscular disorders such as restless leg syndrome and muscle paralysis or tetanus; neuroprotection against stroke, neural trauma and multiple sclerosis; and channelopathies such as erythromyalgia and familial rectal pain syndrome.

As used herein “combination” refers to any mixture or permutation of one or more compounds of the invention and one or more other compounds of the invention or one or more additional therapeutic agent. Unless the context makes clear otherwise, “combination” may include simultaneous or sequentially delivery of a compound of the invention with one or more therapeutic agents. Unless the context makes clear otherwise, “combination” may include dosage forms of a compound of the invention with another therapeutic agent. Unless the context makes clear otherwise, “combination” may include routes of administration of a compound of the invention with another therapeutic agent. Unless the context makes clear otherwise, “combination” may include formulations of a compound of the invention with another therapeutic agent. Dosage forms, routes of administration and pharmaceutical compositions include, but are not limited to, those described herein.

### Kits-of-Parts

The present invention also provides kits that contain a pharmaceutical composition which includes one or more compounds of the invention. The kit also includes instructions for the use of the pharmaceutical composition for modulating the activity of ion channels, for the treatment of pain, as well as other utilities as disclosed herein. Preferably, a commercial package will contain one or more unit doses of the pharmaceutical composition. For example, such a unit dose may be an amount sufficient for the preparation of an intravenous injection. It will be evident to those of ordinary skill in the art that compounds which are light and/or air sensitive may require special packaging and/or formulation. For example, packaging may be used which is opaque to light, and/or sealed from contact with ambient air, and/or formulated with suitable coatings or excipients.

### Preparation of the Compounds of the Invention

The following Reaction Schemes illustrate methods to make compounds of the invention, i.e., compounds of formula (I), compounds of formula (II) and compounds of formula (III), as described above in the Summary of the Invention.

The compounds of the invention may also be prepared according to methods similar to those described in PCT Published Patent Application WO 2006/110917 and to those described in PCT Published Patent Application WO 2008/046049, the disclosures of which are both incorporated in full herein in their entireties, particularly with respect to the methods of preparation disclosed therein for the compounds disclosed therein.

It is also understood that one skilled in the art would be able to make in a similar manner as described below the compounds of the invention by reference to the disclosures of PCT Published Patent Application WO 2006/110917 and to the disclosures of PCT Published Patent Application WO 2008/046049 by using the appropriate starting materials and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc., or synthesized according to sources known to those skilled in the art (see, e.g., Smith, M. B. and J. March, Advanced Organic Chemistry: Reactions, Mechanisms, and Structure, 5th edition (Wiley, December 2000)), or may be prepared as described above in PCT Published Patent Application WO 2006/110917 or in PCT Published Patent Application WO 2008/046049, or may be prepared by methods disclosed herein.

Protecting groups may be added or removed in the preparation of the compounds of the invention in accordance with standard techniques, which are known to one skilled in the art and as described herein. The use of protecting groups is described in detail in Greene, T.W. and P.G.M. Wuts, Greene's Protective Groups in Organic Synthesis (2006), 4^{th} Ed., Wiley. The protecting group may also be a polymer resin such as a Wang resin or a 2-chlorotrityl-chloride resin.

It will also be appreciated by those skilled in the art, although such protected derivatives of compounds of this invention may not possess pharmacological activity as such, they may be administered to a mammal and thereafter metabolized in the body to form compounds of the invention which are pharmacologically active. Such derivatives may therefore be described as “prodrugs”. All prodrugs of compounds of this invention are included within the scope of the invention.

All of the compounds described below as being prepared which may exist in free base or acid form may be converted to their pharmaceutically acceptable salts by treatment with the appropriate inorganic or organic base or acid. Salts of the compounds prepared below may be converted to their free base or acid form by standard techniques. It is understood that all polymorphs, amorphous forms, anhydrates, hydrates, solvates and salts of the compounds of the invention are intended to be within the scope of the invention. Furthermore, all compounds of the invention which contain an acid or an ester group can be converted to the corresponding ester or acid, respectively, by methods known to one skilled in the art or by methods described herein.

### Preparation of Compounds of Formula (Ia) and Compounds of Formula (Ib)

Compounds of formula (Ia) and compounds of formula (Ib) are compounds of formula (I), as set forth above, and are prepared according to the method disclosed below in Reaction Scheme 1, wherein R¹ and R³ are as defined in the Summary of the Invention for compounds of formula (I); and X is chloro, bromo, iodo or tosylate:

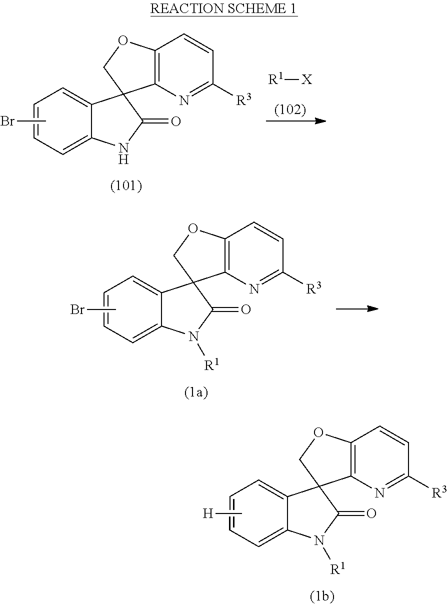

Compounds of formula (101) can be prepared according to the methods disclosed in PCT Published Patent Application No. WO 2008/046049, which is incorporated in full by reference herein. Compounds of formulas (102) are commercially available or can be prepared by methods known to one skilled in the art.

In general, compounds of formula (Ia) and compounds of formula (Ib) are prepared according to the method described above in Reaction Scheme 1 by first treating a compound of formula (101) with an alkylating reagent of formula (102) in the presence of a base such as, but not limited to, sodium hydride, sodium bis(trimethylsilyl)amide, lithium hydroxide or cesium carbonate, in a solvent such as, but not limited to N,N-dimethylformamide, tetrahydrofuran, 2-butanone, acetone, acetonitrile or the combination of any two of them, in the presence or absence of potassium iodide in order to generate a compound of formula (Ia).

The compound of formula (la) can be treated with a palladium catalyst such as, but not limited to, tetrakis(triphenylphosphine)palladium(0) or palladium acetate, formic acid and triethylamine in a solvent such as, but not limited to, dioxane to provide the compound of formula (Ib).

### Preparation of Compounds of Formula (II)

Compounds of formula (II) wherein R⁶ is hydrogen, as described above in the Summary of the Invention, are prepared as set forth below in Reaction Scheme 2, wherein m, R⁴ and R⁵ are as described above in the Summary of the Invention for compounds of formula (II):

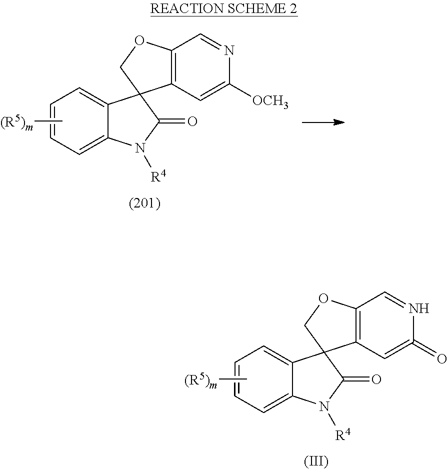

Compounds of formula (201) can be prepared according to the methods disclosed in PCT Published Patent Application No. WO 2008/046049, which is incorporated in full by reference herein. The methoxy compound of formula (201) can be treated with iodotrimethylsilane in a solvent such as, but not limited to, acetonitrile to provide the compound of formula (II).

### Preparation of Compounds of Formula (IIIa), Compounds of Formula (IIIb) and Compounds of Formula (IIIc)

Compounds of formula (IIIa), Compounds of formula (IIIb) and compounds of formula (IIIc) are compounds of formula (III) wherein q is 1, as set forth above in the Summary of the Invention, and are prepared as set forth below in Reaction Scheme 3, wherein J, K, R⁷ and R⁸ are as defined in the Summary of the Invention for compounds of formula (III); PG is a nitrogen-protecting group, such as diphenylmethyl; and X is chloro, bromo, iodo or tosylate:

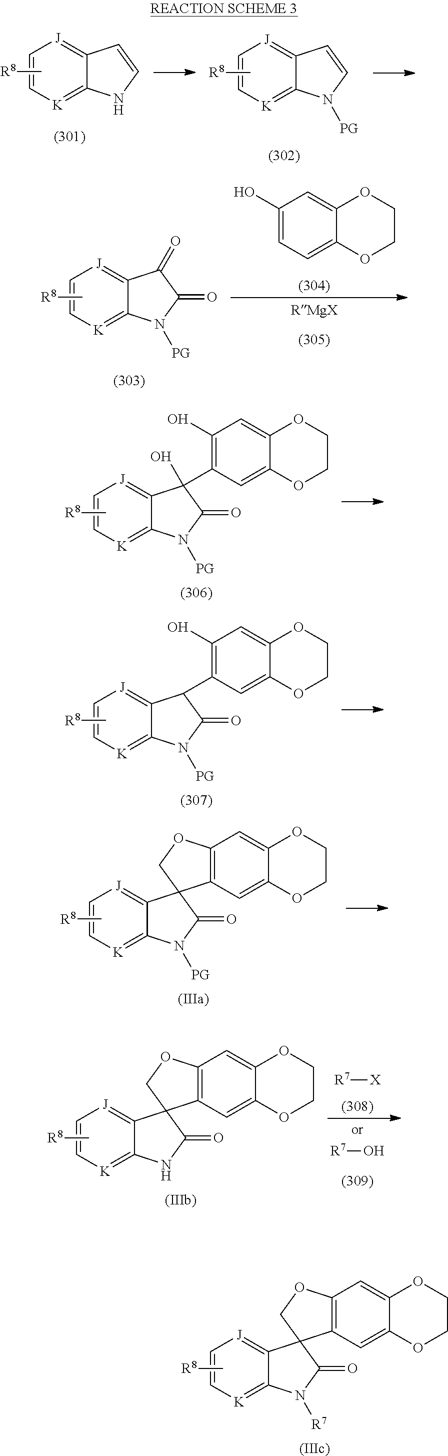

Compounds of formula (301), formula (304), formula (305), formula (308) and formula (309) are commercially available or can be prepared according to methods known to one skilled in the art.

In general, compounds of formula (IIIa), compounds of formula (IIIb) and compounds of formula (IIIc) are prepared according to the method described above in Reaction Scheme 3 by protecting the heteroaryl-fused pyrrole compound of formula (301) to form the compound of formula (302). Compound of formula (302) is then converted to the dicarbonyl compound of formula (303) by treatment with a brominating agent such as, but not limited to, pyridinium tribromide followed by reaction with silver nitrite. The phenol compound of formula (304) is treated with a Grignard reagent of formula (305) at low temperature (0° C.) to form a phenoxymagnesium halide intermediate which reacts with the keto-carbonyl group of the isatin-like compound of formula (303) in a solvent such as, but not limited to, methylene chloride or tetrahydrofuran, to afford the oxindole compound of formula (306). The compound of formula (307) is obtained after the removal of the hydroxyl group at C-3 position of the oxindole by treating the compound of formula (306) with a silane such as, but not limited to, triethylsilane. Compound of formula (307) is treated with an alkylating reagent such as, but not limited to, chloroiodomethane with a base such as, but not limited to, cesium carbonate, in a solvent such as, but not limited to, tetrahydrofuran or N,N-dimethylformamide to afford the compound of formula (IIIa) of the invention via intramolecular cyclization. When PG is diphenylmethyl, it can be removed by the treatment of compound of formula (IIIa) with triethyl silane and trifluoroacetic acid at reflux to provide the compound of formula (IIIb) of the invention where R⁷ is H. The formation of the compound of formula (IIIc) is achieved by alkylation of the compound of formula (IIIb) with an alkylating reagent of formula (308) (where X is chloro, bromo, iodo, or tosylate) in the presence of a base such as, but not limited to, sodium hydride, sodium bis(trimethylsilyl)amide, lithium hydroxide, or cesium carbonate, in a solvent such as, but not limited to, N,N-dimethylformamide, tetrahydrofuran, 2-butanone, acetone, acetonitrile or the combination of any two of them, in the presence or absence of potassium iodide. Alternatively, reaction of compound of formula (IIIb) with an alcohol of formula (309) under Mitsunobu reaction conditions in the presence of a phosphine reagent such as, but not limited to, triphenylphosphine, tributylphosphine, or trimethyl phosphine, and azadicarboxylate of diethyl, diisopropyl, di-tert-butyl or N,N,N′,N′-tetramethylazodicarboxamide in a solvent such as, but not limited to, tetrahydrofuran, ethyl acetate, or dichloromethane, provides the compound of formula (IIIc).

All compounds of the invention which exist in free base or acid form can be converted to their pharmaceutically acceptable salts by treatment with the appropriate inorganic or organic base or acid by methods known to one of ordinary skill in the art. Salts of the compounds of the invention can be converted to their free base or acid form by standard techniques known to one skilled in the art.

The following Preparations, which are directed to the preparation of intermediates used in the preparation of the compounds of the invention, the following Examples, which are directed to the preparation of the compounds of the invention, and the following Biological Examples are provided as a guide to assist in the practice of the invention, and are not intended as a limitation on the scope of the invention.

### PREPARATION 1

### Synthesis of 1-(diphenylmethyl)-3-(7-hydroxy-2,3-dihydro-1,4-benzodioxin-6-yl)-1,3-dihydro-2H-pyrrolo[3,2-b]pyridin-2-one

### A. Synthesis of 1-(diphenylmethyl)-1H-pyrrolo[3,2-b]pyridine

To a solution of 1H-pyrrolo[3,2-b]pyridine (10.0 g, 84.7 mmol) in N,N-dimethylformamide (100 mL) was added sodium hydride (60% w/w dispersion in mineral oil, 2.24 g, 65.3 mmol) in small portions at 0° C. The reaction mixture was stirred at ambient temperature for 1 h and a solution of bromodiphenylmethane (22.0 g, 88.9 mmol) in N,N-dimethylformamide (50 mL) was added dropwise at 0° C. The reaction mixture was stirred at ambient temperature for 17 h and water (400 mL) was added at 0° C. The mixture was filtered and the filter cake was washed with ethyl acetate (3×100 mL). The filtrate was transferred to a separatory funnel and the aqueous layer was extracted with ethyl acetate (3×200 mL). The combined organic extracts were washed with water and brine, dried over anhydrous sodium sulfate, filtered and concentrated in vacuo. The residue was purified by column chromatography, and eluted with a 10% to 50% gradient of ethyl acetate in petroleum ether to afford 1-(diphenylmethyl)-1H-pyrrolo[3,2-b]pyridine (4.70 g, 20%) as a colorless solid: ¹H NMR (400 MHz, CD₃OD) δ 8.30-8.29 (m, 1H), 7.70 (d, J=6.3 Hz, 1H), 7.37-7.04 (m, 13H), 6.62 (d, J=2.4 Hz, 1H).

### B. Synthesis of 3,3-dibromo-1-(diphenylmethyl)-1,3-dihydro-2H-pyrrolo[3,2-b]pyridin-2-one

To a solution of 1-(diphenylmethyl)-1H-pyrrolo[3,2-b]pyridine (4.70 g, 16.5 mmol) in tert-butanol (212 mL) and water (2.8 mL) was added pyridinium tribromide (16.7 g, 52.2 mmol) in small portions at ambient temperature. The reaction mixture was stirred at ambient temperature for 5 h and filtered. The filtrate was concentrated in vacuo and the residue was diluted with ethyl acetate, washed with water and brine, dried over anhydrous sodium sulfate, filtered and concentrated in vacuo. The residue was purified by column chromatography, and eluted with a 10% to 50% gradient of ethyl acetate in petroleum ether to afford 3,3-dibromo-1-(diphenylmethyl)-1,3-dihydro-2H-pyrrolo[3,2-b]pyridin-2-one (5.40 g, 71%) as a yellow solid: MS (ES+) m/z 456 (M+1), 458 (M+1), 460 (M+1).

### C. Synthesis of 1-(diphenylmethyl)-1H-pyrrolo[3,2-b]pyridine-2,3-dione

To a stirred solution of 3,3-dibromo-1-(diphenylmethyl)-1,3-dihydro-2H-pyrrolo[3,2-b]pyridin-2-one (35.2 g, 76.8 mmol) in acetonitrile (1000 mL) and water (67 mL) was added silver nitrite (26.1 g, 153 mmol). The reaction mixture was heated at reflux for 5 h then cooled to ambient temperature. The pH of the mixture was adjusted to ˜8 by the addition of saturated aqueous sodium bicarbonate and the mixture was extracted with ethyl acetate. The combined organic extracts were washed with water and brine, dried over anhydrous sodium sulfate, filtered and concentrated in vacuo. The residue was purified by column chromatography, and eluted with a 10% to 50% gradient of ethyl acetate in petroleum ether to afford 1-(diphenylmethyl)-1H-pyrrolo[3,2-b]pyridine-2,3-dione (10.0 g, 41%) as a red solid: ¹H NMR (400 MHz, DMSO-d₆) δ 8.31 (d, J=2.8 Hz, 1H), 7.37-7.31 (m, 11H), 6.82 (s, 1H).

### D. Synthesis of 1-(diphenylmethyl)-3-hydroxy-3-(7-hydroxy-2,3-dihydro-1,4-benzodioxin-6-yl)-1,3-dihydro-2H-pyrrolo[3,2-b]pyridin-2-one

To a solution of 2,3-dihydrobenzo[b][1,4]dioxin-6-ol (0.45 g, 2.7 mmol) in tetrahydrofuran (18 mL) was added isopropylmagnesium chloride (2.0 M solution in tetrahydrofuran, 1.6 mL, 3.2 mmol) dropwise at 0° C. The reaction mixture was stirred at 0° C. for 0.5 h and concentrated in vacuo. The residue was dissolved in dichloromethane (10 mL) and a solution of 1-(diphenylmethyl)-1H-pyrrolo[3,2-b]pyridine-2,3-dione (0.77 g, 2.5 mmol) in dichloromethane (5 mL) was added at 0° C. The reaction mixture was stirred at ambient temperature for 3 h and saturated aqueous ammonium chloride (15 mL) was added. The phases were separated and the aqueous phase was extracted with dichloromethane (2×150 mL). The combined organic extracts were washed with water and brine, dried over anhydrous sodium sulfate, filtered and concentrated in vacuo. The residue was purified by column chromatography, and eluted with a 10% to 50% gradient of ethyl acetate in petroleum ether to afford 1-(diphenylmethyl)-3-hydroxy-3-(7-hydroxy-2,3-dihydro-1,4-benzodioxin-6-yl)-1,3-dihydro-2H-pyrrolo[3,2-b]pyridin-2-one (0.95 g, 79%) as a yellow solid: ¹H NMR (400 MHz, CD₃OD) δ 7.84-7.82 (m, 1H), 7.27-7.21 (m, 11H), 6.90 (s, 1H), 6.87-6.84 (m, 1H), 6.60-6.58 (m, 1H), 6.11 (s, 1H), 4.09-4.06 (m, 4H).

### E. Synthesis of 1-(diphenylmethyl)-3-(7-hydroxy-2,3-dihydro-1,4-benzodioxin-6-yl)-1,3-dihydro-2H-pyrrolo[3,2-b]pyridin-2-one

To a solution of 1-(diphenylmethyl)-3-hydroxy-3-(7-hydroxy-2,3-dihydro-1,4-benzodioxin-6-yl)-1,3-dihydro-2H-pyrrolo[3,2-b]pyridin-2-one (3.50 g, 7.35 mmol) and triethylsilane (10.0 mL, 62.6 mmol) in dichloromethane (60 mL) was added trifluoroacetic acid (55.0 mL, 103 mmol) at ambient temperature. The reaction mixture was heated at 70° C. for 17 h, cooled to ambient temperature and concentrated in vacuo. The residue was purified by column chromatography, and eluted with a 10% to 33% gradient of ethyl acetate in petroleum ether to afford 1-(diphenylmethyl)-3-(7-hydroxy-2,3-dihydro-1,4-benzodioxin-6-yl)-1,3-dihydro-2H-pyrrolo[3,2-b]pyridin-2-one (2.60 g, 77%) as a yellow solid: ¹H NMR (400 MHz, CD₃OD) δ 7.39-7.29 (m, 8H), 7.24-7.21 (m, 5H), 7.09 (s, 1H), 6.59-6.57 (m, 1H), 6.46 (s, 1H), 6.41-6.37 (m, 1H), 4.24-4.20 (m, 4H).

### PREPARATION 2

### Synthesis of 1-(diphenylmethyl)-3-(7-hydroxy-2,3-dihydro-1,4-benzodioxin-6-yl)-1,3-dihydro-2H-pyrrolo[2,3-b]pyridin-2-one

### A. Synthesis of 1-(diphenylmethyl)-1H-pyrrolo[2,3-b]pyridine

Following the procedure as described in PREPARATION 1A, and making non-critical variations using 1H-pyrrolo[2,3-b]pyridine to replace 1H-pyrrolo[3,2-b]pyridine, 1-(diphenylmethyl)-1H-pyrrolo[2,3-b]pyridine (30%) was obtained as a colorless solid: ¹H NMR (400 MHz, CDCl₃) δ 8.34-8.32 (m, 1H), 7.96-7.93 (m, 1H), 7.53 (d, J=3.6 Hz, 1H), 7.39-7.26 (m, 6H), 7.17-7.07 (m, 6H), 6.49 (d, J=3.6 Hz, 1H); MS (ES+) m/z 285 (M+1).

### B. Synthesis of 3,3-dibromo-1-(diphenylmethyl)-1,3-dihydro-2H-pyrrolo[2,3-b]pyridin-2-one

To a solution of 1-(diphenylmethyl)-1H-pyrrolo[2,3-b]pyridine (42.6 g, 0.15 mol) in tert-butanol (2500 mL) was added pyridinium tribromide (140 g, 0.44 mol) in small portions at ambient temperature. The reaction mixture was stirred at 40° C. for 3 h. Further pyridinium tribromide (32.0 g, 0.10 mol) was added at ambient temperature, and the mixture was stirred at 40° C. for 2 h. The reaction mixture was cooled to 0° C. and water (1000 mL) was added. The mixture was extracted with ethyl acetate (3×500 mL) and the combined organic extracts was washed with water and brine, dried over anhydrous sodium sulfate, filtered and concentrated in vacuo to afford 3,3-dibromo-1-(diphenylmethyl)-1,3-dihydro-2H-pyrrolo[2,3-b]pyridin-2-one (73.5 g): MS (ES+) m/z 456 (M+1), 458 (M+1), 460 (M+1).

### C. Synthesis of 1-(diphenylmethyl)-1H-pyrrolo[2,3-b]pyridine-2,3-dione

Following the procedure as described in PREPARATION 1C, and making non-critical variations using 3,3-dibromo-1-(diphenylmethyl)-1,3-dihydro-2H-pyrrolo[2,3-b]pyridin-2-one to replace 3,3-dibromo-1-(diphenylmethyl)-1,3-dihydro-2H-pyrrolo[3,2-b]pyridin-2-one, 1-(diphenylmethyl)-1H-pyrrolo[2,3-b]pyridine-2,3-dione (41%) was obtained as a yellow solid: MS (ES+) m/z 315 (M+1).

### D. Synthesis of 1-(diphenylmethyl)-3-hydroxy-3-(7-hydroxy-2,3-dihydro-1,4-benzodioxin-6-yl)-1,3-dihydro-2H-pyrrolo[2,3-b]pyridin-2-one

Following the procedure as described in PREPARATION 1D, and making non-critical variations using 1-(diphenylmethyl)-1H-pyrrolo[2,3-b]pyridine-2,3-dione to replace 1-(diphenylmethyl)-1H-pyrrolo[3,2-b]pyridine-2,3-dione, 1-(diphenylmethyl)-3-hydroxy-3-(7-hydroxy-2,3-dihydro-1,4-benzodioxin-6-yl)-1,3-dihydro-2H-pyrrolo[2,3-b]pyridin-2-one (79%) was obtained as a yellow solid: ¹H NMR (400 MHz, DMSO-d₆) δ 9.05 (s, 1H), 8.01-7.99 (m, 1H), 7.44-7.21 (m, 12H), 6.90-6.87 (m, 1H), 6.81 (s, 1H), 6.69 (s, 1H), 6.17 (s, 1H), 4.19-4.18 (m, 4H); MS (ES+) m/z 467 (M+1).

### E. Synthesis of 1-(diphenylmethyl)-3-(7-hydroxy-2,3-dihydro-1,4-benzodioxin-6-yl)-1,3-dihydro-2H-pyrrolo[2,3-b]pyridin-2-one

To a stirred solution of 1-(diphenylmethyl)-3-hydroxy-3-(7-hydroxy-2,3-dihydro-1,4-benzodioxin-6-yl)-1,3-dihydro-2H-pyrrolo[2,3-b]pyridin-2-one (26.0 g, 55.8 mmol) and triethylsilane (72.0 mL, 450 mmol) in dichloromethane (500 mL) was added trifluroacetic acid (55.0 mL, 714 mmol) at ambient temperature. The reaction mixture was heated at reflux for 12 h, cooled to ambient temperature and concentrated in vacuo. The residue was purified by column chromatography, and eluted with ethyl acetate/petroleum ether (1/4) to afford 1-(diphenylmethyl)-3-(7-hydroxy-2,3-dihydro-1,4-benzodioxin-6-yl)-1,3-dihydro-2H-pyrrolo[2,3-b]pyridin-2-one (14.0 g, 56%) as a yellow solid: ¹H NMR (400 MHz, DMSO-d₆) δ 9.15 (s, 1H), 8.10 (d, J=5.2 Hz, 1H), 7.43-7.21 (m, 12H), 6.93-6.88 (m, 2H), 6.68 (s, 1H), 6.27 (s, 1H), 4.84 (s, 1H), 4.17-4.13 (m, 4H); MS (ES+) m/z 451 (M+1).

### EXAMPLE 1

### Synthesis of 4′-bromo-5-methoxy-1′-[(2R)-tetrahydrofuran-2-ylmethyl]spiro[furo[3,2-b]pyridine-3,3′-indol]-2′(1′H)-one

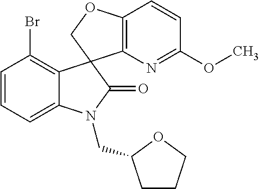

To a stirred solution of 4′-bromo-5-methoxyspiro[furo[3,2-b]pyridine-3,3′-indol]-2′(1′H)-one (prepared by the methods described in PCT Published Patent Application WO 2008/046049) (1.15 g, 3.3 mmol) in 2-butanone (40 mL) was added cesium carbonate (3.2 g, 9.9 mmol) and (R)-(tetrahydrofuran-2-yl)methyl 4-methylbenzenesulfonate (1.06 g, 4.2 mmol). The reaction was heated at reflux for 4 h, cooled to ambient temperature and filtered. The filtrate was concentrated in vacuo and the residue was purified by column chromatography, and eluted with a 20% to 50% gradient of ethyl acetate in hexanes to afford 4′-bromo-5-methoxy-1′-[(2R)-tetrahydrofuran-2-ylmethyl]spiro[furo[3,2-b]pyridine-3,3′-indol]-2′(1′H)-one (1.14 g, 80%) as a colorless solid: mp 110-112° C.; ¹H NMR (300 MHz, DMSO-d₆) δ 7.35 (dd, J=8.8, 1.0 Hz, 1H), 7.30-7.13 (m, 3H), 6.65 (d, J=8.9 Hz, 1H), 4.94 (d, J=10.0 Hz, 1H), 4.78-4.72 (m, 1H), 4.19-4.06 (m, 1H), 3.93-3.54 (m, 4H), 3.52 (d, J=3.3 Hz, 3H), 1.97-1.52 (m, 4H); ¹³C NMR (75 MHz, DMSO-d₆) δ 176.4 (2C), 159.7 (2C), 151.1, 146.1 (2C), 143.6 (2C), 131.2 (2C), 129.2 (2C), 126.5 (2C), 121.5 (2C), 118.7 (2C), 110.7 (2C), 109.9 (2C), 76.6 (2C), 76.1 (2C), 67.8, 53.7 (2C), 44.4 (2C), 28.7 (2C), 25.6 (2C); MS (ES+) m/z 431.0 (M+1), 433.0 (M+1).

### EXAMPLE 2

### Synthesis of 5-methoxy-1′-[(2R)-tetrahydrofuran-2-ylmethyl]spiro[furo[3,2-b]pyridine-3,3′-indol]-2′(1′H)-one

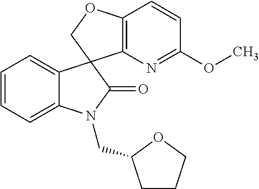

A mixture of 4′-bromo-5-methoxy-1′-[(2R)-tetrahydrofuran-2-ylmethyl]spiro[furo[3,2-b]pyridine-3,3′-indol]-2′(1′H)-one (0.60 g, 1.4 mmol), tetrakis(triphenylphosphine)palladium(0) (0.33 g, 0.28 mmol), formic acid (0.7 mL, 18 mmol) and triethylamine (1.8 g, 18 mmol) and p-dioxane (25 mL) was heated at reflux for 16 h, cooled to ambient temperature and concentrated in vacuo. The residue was purified by column chromatography, and eluted with a 30% to 60% gradient of ethyl acetate in hexanes to afford 5-methoxy-1-[(2R)-tetrahydrofuran-2-ylmethyl]spiro[furo[3,2-b]pyridine-3,3′-indol]-2′(1′H)-one (0.20 g, 40%) as a colorless solid: mp 52-56° C.; ¹H NMR (300 MHz, CDCl₃) δ 7.33-6.97 (m, 5H), 6.53 (d, J=8.8 Hz, 1H), 4.99 (dd, J=9.2, 1.0 Hz, 1H), 4.72 (d, J=9.2 Hz, 1H), 4.35-4.23 (m, 1H), 4.05-3.64 (m, 4H), 3.64, 3.62 (s, 3H), 2.07-1.74 (m, 4H); ¹³C NMR (75 MHz, CDCl₃) δ 177.0 (2C), 160.2 (2C), 149.4 (2C), 145.6 (2C), 143.5 (2C), 142.9 (2C), 131.4 (2C), 128.9 (2C), 123.4 (2C), 123.1 (2C), 121.1 (2C), 110.4 (2C), 109.3, 79.4 (2C), 68.3 (2C), 58.8, 53.6 (2C), 44.4 (2C), 28.8 (2C), 25.8 (2C); MS (ES+) m/z 353.1 (M+1).

### EXAMPLE 3

### Synthesis of 1′-(2,3-dihydro-1,4-benzodioxin-6-ylmethyl)-5-methoxyspiro[furo[3,2-b]pyridine-3,3′-indol]-2′(1′H)-one

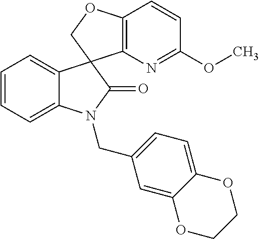

A mixture of 5-methoxyspiro[furo[3,2-b]pyridine-3,3′-indol]-2′(1′H)-one (prepared as described in PCT Published Patent Application WO 2008/046049) (0.11 g, 0.40 mmol), 6-(bromomethyl)-2,3-dihydrobenzo[b][1,4]dioxane (prepared as described in Capilla, A. S. et al. Tetrahedron (2001), 57:8297-304) (0.12 g, 0.53 mmol), cesium carbonate (0.21 g, 0.63 mmol) and acetonitrile (3.5 mL) was stirred at ambient temperature for 17 h. The mixture was diluted with ethyl acetate and filtered through a pad of diatomaceous earth. The filtrate was concentrated in vacuo and the residue was purified by column chromatography, eluted with hexanes/ethyl acetate (3/1) to afford 1′-(2,3-dihydro-1,4-benzodioxin-6-ylmethyl)-5-methoxyspiro[furo[3,2-b]pyridine-3,3′-indol]-2′(1′H)-one (0.17 g, quantitative yield) as a colorless solid: mp 144-146° C. (methanol/water); ¹H NMR (300 MHz, CDCl₃) δ 7.23-7.14 (m, 3H), 7.02 (dd, J=7.5, 7.5 Hz, 1H), 6.94-6.89 (m, 2H), 6.79-6.74 (m, 2H), 6.56 (d, J=8.7 Hz, 1H), 5.26 (d, J=15.8 Hz, 1H), 5.09 (d, J=9.3 Hz, 1H), 4.81 (d, J=9.3 Hz, 1H), 4.57 (d, J=15.8 Hz, 1H), 4.21 (s, 4H), 3.73 (s, 3H); ¹³C NMR (75 MHz, CDCl₃) δ 176.5, 160.4, 149.3, 145.8, 143.9, 143.1, 142.5, 131.8, 129.0, 128.6, 123.6, 123.3, 121.3, 120.1, 117.6, 116.0, 110.7, 109.7, 79.2, 64.4, 59.1, 54.0, 43.5; MS (ES+) m/z 416.9 (M+1).

### EXAMPLE 4

### Synthesis of 1′-[(2R)-tetrahydrofuran-2-ylmethyl]spiro[furo[2,3-c]pyridine-3,3′-indole]-2′,5(1′H,6H)-dione

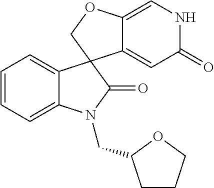

To a solution of 5-methoxy-1′-[(2R)-tetrahydrofuran-2-ylmethyl]spiro[furo[2,3-c]pyridine-3,3′-indol]-2′(1′H)-one (prepared as described in PCT Published Patent Application WO 2008/046049) (1.78 g, 5.1 mmol) in anhydrous acetonitrile (25 mL) was added iodotrimethylsilane (1.4 mL, 10 mmol). The reaction mixture was heated at reflux for 4 h and cooled to ambient temperature. The reaction mixture was concentrated in vacuo and the residue purified by column chromatography, and eluted with a 0% to 10% gradient of methanol in dichloromethane to afford 1′-[(2R)-tetrahydrofuran-2-ylmethyl]spiro[furo[2,3-c]pyridine-3,3′-indole]-2′,5(1′H,6H)-dione (1.59 g, 93%) as an orange solid: mp>250° C. (dichloromethane/methanol); ¹H NMR (300 MHz, DMSO-d₆) δ 7.38-7.33 (m, 1H), 7.30-7.16 (m, 3H), 7.10-7.03 (m, 1H), 5.88 (d, J=5.9 Hz, 1H), 4.78-4.62 (m, 2H), 3.88-3.54 (m, 3H), 3.32-3.23 (m, 2H), 2.02-1.78 (m, 2H), 1.63-1.33 (m, 2H); ¹³C NMR (75 MHz, DMSO-d₆) δ 175.0, 174.9, 143.7, 143.4, 143.1, 129.3, 129.1, 123.5, 123.4, 123.0, 122.8, 110.1 (2C), 79.4, 75.7, 75.5, 67.3, 67.2, 66.7, 66.3, 57.0, 48.6, 46.7, 46.4, 44.0, 43.9, 43.8, 35.2 (2C), 29.5, 29.4, 28.7, 28.5, 25.1 (2C); MS (ES+) m/z 338.9 (M+1).

### EXAMPLE 5

### Synthesis of 1′-(diphenylmethyl)-2,3-dihydrospiro[furo[2,3-g][1,4]benzodioxine-8,3′-pyrrolo[2,3-b]pyridin]-2′(1′H)-one

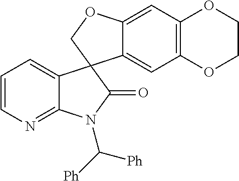

To a solution of 1-(diphenylmethyl)-3-(7-hydroxy-2,3-dihydro-1,4-benzodioxin-6-yl)-1,3-dihydro-2H-pyrrolo[2,3-b]pyridin-2-one (23.1 g, 51.3 mmol) and chloroiodomethane (27.5 g, 156 mmol) in N,N-dimethylformamide (500 mL) was added cesium carbonate (83.7 g, 257 mmol). The reaction mixture was stirred at ambient temperature for 12 h and filtered through a pad of diatomaceous earth. The filtrate was extracted with ethyl acetate (3×500 mL) and the combined organic extracts were washed with water and brine, dried over anhydrous sodium sulfate, filtered and concentrated in vacuo. The residue was purified by column chromatography, and eluted with ethyl acetate/petroleum ether (1/6) to afford 1′-(diphenylmethyl)-2,3-dihydrospiro[furo[2,3-g][1,4]benzodioxine-8,3′-pyrrolo[2,3-b]pyridin]-2′(1′H)-one (16.0 g, 67%): MS (ES+) m/z 463 (M+1).

### EXAMPLE 6

### Synthesis of 2,3-dihydrospiro[furo[2,3-g][1,4]benzodioxine-8,3′-pyrrolo[2,3-b]pyridin]-2′(1′H)-one

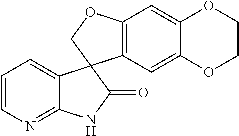

To a solution of 1′-(diphenylmethyl)-2,3-dihydrospiro[furo[2,3-g][1,4]benzodioxine-8,3′-pyrrolo[2,3-b]pyridin]-2′(1′H)-one (18.0 g, 39.0 mmol) in trifluoroacetic acid (150 mL) was added triethylsilane (30.0 mL). The reaction mixture was heated at reflux for 12 h, cooled to ambient temperature and concentrated in vacuo. The residue was triturated in diethyl ether to afford 2,3-dihydrospiro[furo[2,3-g][1,4]benzodioxine-8,3′-pyrrolo[2,3-b]pyridin]-2′(1′H)-one (7.90 g, 69%) as a colorless solid: mp>250° C.; ¹H NMR (300 MHz, DMSO-d₆) δ 11.16 (s, 1H), 8.10-8.08 (m, 1H), 7.47-7.44 (m, 1H), 6.96-6.92 (m, 1H), 6.46 (s, 1H), 6.24 (s, 1H), 4.66 (ABq, 2H), 4.15-4.07 (m, 4H); MS (ES+) m/z 296.8 (M+1).

### EXAMPLE 7

### Synthesis of 1′-(pyridin-2-ylmethyl)-2,3-dihydrospiro[furo[2,3-g][1,4]benzodioxine-8,3′-pyrrolo[2,3-b]pyridin]-2′(1′H)-one

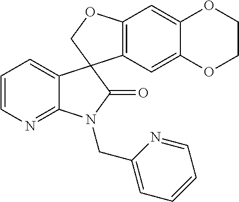

To a solution of 2,3-dihydrospiro[furo[2,3-g][1,4]benzodioxine-8,3′-pyrrolo[2,3-b]pyridin]-2′(1′H)-one (0.30 g, 1.0 mmol) in anhydrous N,N-dimethylformamide (15 mL) were added cesium carbonate (1.63 g, 5.0 mmol), potassium iodide (0.05 g, 0.3 mmol), and 2-(bromomethyl)pyridine hydrobromide (0.29 g, 1.2 mmol) at ambient temperature. The reaction mixture was heated at 95° C. for 2 h and cooled to ambient temperature. Further, potassium iodide (0.05 g, 0.3 mmol) and cesium carbonate (0.33 g, 1.0 mmol) were added. The reaction mixture was heated at 110° C. for 4 h, cooled to ambient temperature and concentrated in vacuo. The solid residue was treated with water (150 mL), sonicated, and filtered. The solid was purified by column chromatography, and eluted with a 0% to 100% gradient of ethyl acetate in dichloromethane to afford 1′-(pyridin-2-ylmethyl)-2,3-dihydrospiro[furo[2,3-g][1,4]benzodioxine-8,3′-pyrrolo[2,3-b]pyridin]-2′(1′H)-one (0.12 g, 33%) as a colorless solid: ¹H NMR (300 MHz, DMSO-d₆) δ 8.40 (d, J=4.7 Hz, 1H), 8.10 (dd, J=5.2, 1.2 Hz, 1H), 7.75 (ddd, J=7.6, 7.6, 1.6 Hz, 1H), 7.57 (dd, J=7.3, 1.2 Hz, 1H), 7.38-7.22 (m, 2H), 7.01 (dd, J=7.2, 5.3 Hz, 1H), 6.49 (s, 1H), 6.45 (s, 1H), 5.04 (s, 2H), 4.76 (ABq, 2H), 4.20-4.05 (m, 4H); ¹³C NMR (75 MHz, DMSO-d₆) δ 177.1, 156.5, 155.6, 155.2, 149.4, 147.8, 144.8, 138.3, 137.4, 132.0, 126.9, 123.0, 121.8, 121.0, 119.5, 112.2, 99.2, 79.3, 64.7, 64.1, 57.5, 44.0; MS (ES+) m/z 388.0 (M+1).

### EXAMPLE 8

### Synthesis of 1′-{[3-(trifluoromethyl)pyridin-2-yl]methyl}-2,3-dihydrospiro[furo[2,3-g][1,4]benzodioxine-8,3′-pyrrolo[2,3-b]pyridin]-2′(1′H)-one

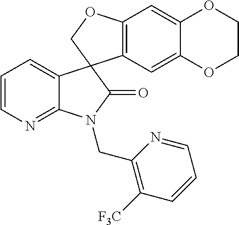

To a solution of 2,3-dihydrospiro[furo[2,3-g][1,4]benzodioxine-8,3′-pyrrolo[2,3-b]pyridin]-2′(1′H)-one (0.30 g, 1.0 mmol) in anhydrous N,N-dimethylformamide (10 mL) at ambient temperature was added cesium carbonate (0.91 g, 2.8 mmol), potassium iodide (0.093 g, 0.6 mmol) and 2-(chloromethyl)-3-(trifluoromethyl)pyridine hydrochloride (prepared by treatment of (3-(trifluoromethyl)pyridin-2-yl)methanol (0.28 g, 1.2 mmol) with thionyl chloride (0.28 g, 1.3 mmol)). The reaction mixture was heated at 95° C. for 2.5 h, cooled to ambient temperature and concentrated in vacuo. The residue was treated with water (200 mL), sonicated and filtered. The solid was purified by column chromatography, and eluted with a 0% to 30% gradient of ethyl acetate in dichloromethane, followed by recrystallization from acetone/hexanes to afford 1′-{[3-(trifluoromethyl)pyridin-2-yl]methyl}-2,3-dihydrospiro[furo[2,3-g][1,4]benzodioxine-8,3′-pyrrolo[2,3-b]pyridin]-2′(1′H)-one (0.11 g, 24%) as a colorless solid: ¹H NMR (300 MHz, DMSO-d₆) δ 8.59 (d, 1H, J=4.5 Hz), 8.22 (d, J=7.7 Hz, 1H), 8.07 (dd, J=5.2, 1.4 Hz, 1H), 7.59 (dd, J=7.3, 1.4 Hz, 1H), 7.53 (dd, J=7.7, 5.0 Hz, 1H), 7.01 (dd, J=7.3, 5.3 Hz, 1H), 6.52 (s, 1H), 6.49 (s, 1H), 5.20 (s, 2H), 4.80-4.73 (m, 2H), 4.18-4.09 (m, 4H); ¹³C NMR (75 MHz, DMSO-d₆) δ 177.2, 156.3, 155.1, 152.7, 152.5, 147.8, 144.8, 138.3, 135.6, 132.1, 126.9, 126.2, 123.5, 123.0, 122.6, 121.0, 119.5, 112.3, 99.2, 79.1, 64.7, 64.1, 57.5; MS (ES+) m/z 456.2 (M+1).

### EXAMPLE 9

### Synthesis of 1′-(diphenylmethyl)-2,3-dihydrospiro[furo[2,3-g][1,4]benzodioxine-8,3′-pyrrolo[3,2-b]pyridin]-2′(1′H)-one

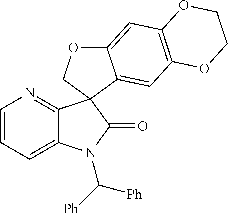

Following the procedure as described in EXAMPLE 5, and making non-critical variations using 1-(diphenylmethyl)-3-(7-hydroxy-2,3-dihydro-1,4-benzodioxin-6-yl)-1,3-dihydro-2H-pyrrolo[3,2-b]pyridin-2-one to replace 1-(diphenylmethyl)-3-(7-hydroxy-2,3-dihydro-1,4-benzodioxin-6-yl)-1,3-dihydro-2H-pyrrolo[2,3-b]pyridin-2-one, 1′-(diphenylmethyl)-2,3-dihydrospiro[furo[2,3-g][1,4]benzodioxine-8,3′-pyrrolo[3,2-b]pyridin]-2′(1′H)-one (84%) was obtained: ¹H NMR (400 MHz, CDCl₃) δ 8.18 (d, J=4.4 Hz, 1H), 7.43-7.23 (m, 10H), 7.09 (s, 1H), 6.99-6.97 (m, 1H), 6.75-6.73 (m, 1H), 6.53 (s, 1H), 6.14 (s, 1H), 5.01 (ABq, 2H), 4.19-4.11 (m, 4H).

### EXAMPLE 10

### Synthesis of 2,3-dihydrospiro[furo[2,3-g][1,4]benzodioxine-8,3′-pyrrolo[3,2-b]pyridin]-2′(1′H)-one

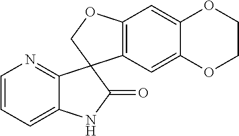

Following the procedure as described in EXAMPLE 6, and making non-critical variations using 1′-(diphenylmethyl)-2,3-dihydrospiro[furo[2,3-g][1,4]benzodioxine-8,3′-pyrrolo[3,2-b]pyridin]-2′(1′H)-one to replace 1′-(diphenylmethyl)-2,3-dihydrospiro[furo[2,3-g][1,4]benzodioxine-8,3′-pyrrolo[2,3-b]pyridin]-2′(1′H)-one, 2,3-dihydrospiro[furo[2,3-g][1,4]benzodioxine-8,3′-pyrrolo[3,2-b]pyridin]-2′(1′H)-one (23%) was obtained: mp>250° C.; ¹H NMR (300 MHz, DMSO-d₆) δ 10.07 (s, 1H), 8.05-7.96 (m, 1H), 7.24-7.09 (m, 2H), 6.50 (s, 1H), 6.17 (s, 1H), 4.68 (ABq, 2H), 5.13-3.77 (m, 4H); MS (ES+) m/z 296.9 (M+1).

### EXAMPLE 11

### Synthesis of 1′-(pyridin-2-ylmethyl)-2,3-dihydrospiro[furo[2,3-g][1,4]benzodioxine-8,3′-pyrrolo[3,2-b]pyridin]-2′(1′H)-one

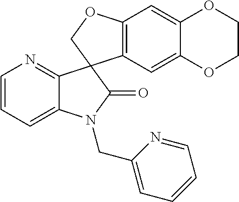

To a solution of 3,7-dihydro-2H-spiro[benzofuro[5,6-b][1,4]dioxine-8,3′-pyrrolo[3,2-b]pyridin]-2′(1′H)-one (0.50 g, 1.7 mmol), 2-pyridylcarbinol (0.28 g, 2.6 mmol) and tri-n-butylphosphine (0.53 g, 2.6 mmol) in tetrahydrofuran (11 mL) was added a solution of diethyl azodicarboxylate (0.45 g, 2.6 mmol) in tetrahydrofuran (6 mL). The solution was stirred at ambient temperature for 16 h and 1 M hydrochloric acid (10 mL) was added. The mixture was washed with diethyl ether (2×50 mL), basified with 5 M aqueous sodium hydroxide (5 mL) and extracted with dichloromethane (3×50 mL). The combined organic extracts were dried over anhydrous magnesium sulfate, filtered and concentrated in vacuo. The residue was purified by column chromatography, and eluted with a 25% to 50% gradient of ethyl acetate in hexanes to afford 1′-(pyridin-2-ylmethyl)-3,7-dihydro-2H-spiro[benzofuro[5,6-b][1,4]dioxine-8,3′-pyrrolo[3,2-b]pyridin]-2′(1′H)-one (0.42 g, 64%) as a colorless solid: mp 146-147° C.; ¹H NMR (300 MHz, CDCl₃) δ 8.57-8.50 (m, 1H), 8.20-8.16 (m, 1H), 7.70-7.62 (m, 1H), 7.34-7.02 (m, 4H), 6.48 (s, 1H), 6.25 (s, 1H), 5.20 (d, J=15.7 Hz, 1H), 4.98-4.85 (m, 3H), 4.17-4.04 (m, 4H); ¹³C NMR (75 MHz, CDCl₃) δ 176.1, 155.7, 154.8, 152.4, 149.6, 144.9, 144.2, 138.2, 137.6, 137.2, 123.4, 123.0, 122.0, 119.4, 116.1, 111.4, 99.8, 78.0, 64.5, 63.9, 58.7, 45.8; MS (ES+) m/z 387.9 (M+1).

### EXAMPLE 12

### Synthesis of 1′-{[3-(trifluoromethyl)pyridin-2-yl]methyl}-2,3-dihydrospiro[furo[2,3-g][1,4]benzodioxine-8,3′-pyrrolo[3,2-b]pyridin]-2′(1′H)-one

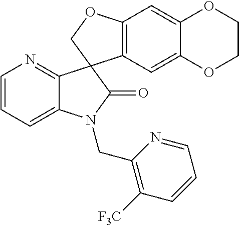

To a solution of 3,7-dihydro-2H-spiro[benzofuro[5,6-b][1,4]dioxine-8,3′-pyrrolo[3,2-b]pyridin]-2′(1′H)-one (0.50 g, 1.7 mmol) in N,N-dimethylformamide (25 mL) were added cesium carbonate (2.2 g, 6.8 mmol), 2-(chloromethyl)-3-(trifluoromethyl)pyridine hydrochloride (0.47 g, 2.6 mmol) and potassium iodide (0.025 g, 0.15 mmol). The mixture was stirred at 100° C. for 16 h, cooled to ambient temperature and poured into water (25 mL). The mixture was extracted with ethyl acetate (3×50 mL) and the combined organic extracts were washed with brine (3×25 mL), dried over anhydrous magnesium sulfate, filtered and concentrated in vacuo. The residue was purified by flash chromatography, and eluted with a 25% to 50% gradient of ethyl acetate in hexanes to afford 1′-((3-(trifluoromethyl)pyridin-2-yl)methyl)-3,7-dihydro-2H-spiro[benzofuro[5,6-b][1,4]dioxine-8,3′-pyrrolo[3,2-b]pyridin]-2′(1′H)-one (0.36 g, 47%) as a colorless solid: mp>250° C.; ¹H NMR (300 MHz, DMSO-d₆) δ 8.66 (d, J=4.4 Hz, 1H), 8.27 (d, J=7.9 Hz, 1H), 8.13 (dd, J=4.9, 1.2 Hz, 1H), 7.59 (dd, J=7.8, 5.0 Hz, 1H), 7.36 (dd, J=8.0, 1.2 Hz, 1H), 7.26 (dd, J=8.0, 5.0 Hz, 1H), 6.49 (s, 1H), 6.45 (s, 1H), 5.38-5.11 (m, 2H), 4.84-4.76 (m, 2H), 4.22-4.08 (m, 4H); ¹³C NMR (75 MHz, DMSO-d₆) δ 176.1, 155.4, 152.9, 152.7, 152.6, 144.7, 143.5, 138.9, 138.1, 135.6, 124.3 (q, ¹J_{C—F}=273 Hz), 124.1, 123.7, 123.5, 120.7, 116.5, 112.2, 100.0, 77.9, 64.7, 64.1, 58.4, 42.3; MS (ES+) m/z 456.0 (M+1).

### EXAMPLE 13

### Synthesis of 5-methoxy-1′-{[3-(trifluoromethyl)pyridin-2-yl]methyl}spiro[furo[3,2-b]pyridine-3,3′-indol]-2′(1′H)-one

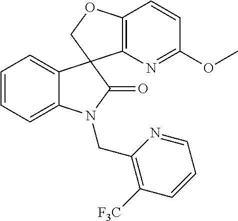

Following the procedure as described in EXAMPLE 7, and making non-critical variations using 5-methoxyspiro[furo[3,2-b]pyridine-3,3′-indol]-2′(1′H)-one to replace 2,3-dihydrospiro[furo[2,3-g][1,4]benzodioxine-8,3′-pyrrolo[2,3-b]pyridin]-2′(1′H)-one, and 2-(chloromethyl)-3-(trifluoromethyl)pyridine hydrochloride to replace 2-(bromomethyl)pyridine hydrobromide, 5-methoxy-1′-{[3-(trifluoromethyl)pyridin-2-yl]methyl}spiro[furo[3,2-b]pyridine-3,3′-indol]-2′(1′H)-one (76%) was obtained as a colorless solid: mp 195-198° C.; ¹H NMR (300 MHz, DMSO-d₆) δ 8.68 (d, J=4.4 Hz, 1H), 8.23 (d, J=7.2 Hz, 1H), 7.54 (dd, J=7.8, 4.9 Hz, 1H), 7.45 (d, J=8.8 Hz, 1H), 7.30 (ddd, J=8.2, 8.2, 5.8 Hz, 1H), 7.23 (d, J=6.6 Hz, 1H), 7.04 (dd, J=7.5 Hz, 1H), 6.94 (d, J=7.8 Hz, 1H), 6.69 (d, J=8.8 Hz, 1H), 5.25 (s, 2H), 4.86 (q, J=9.5 Hz, 2H), 3.61 (s, 3H); ¹³C NMR (75 MHz, DMSO-d₆) δ 176.2, 159.3, 152.6, 152.4, 152.3, 149.5, 145.3, 143.5, 134.7, 130.3, 128.9, 128.8, 125.6 (q, ¹J_{C—F}=274 Hz), 123.7, 122.8, 121.4, 109.9, 109.2, 79.2, 58.0, 53.3, 42.2; MS (ES+) m/z 427.2 (M+1).

### BIOLOGICAL ASSAYS

Various techniques are known in the art for testing the activity of the compound of the invention or determining their solubility in known pharmaceutically acceptable excipients. In order that the invention described herein may be more fully understood, the following biological assays are set forth. It should be understood that these examples are for illustrative purposes only and are not to be construed as limiting this invention in any manner.

### BIOLOGICAL EXAMPLE 1

### Guanidine Influx Assay (In Vitro Assay)

This example describes an in vitro assay for testing and profiling test agents against human or rat sodium channels stably expressed in cells of either an endogenous or recombinant origin. The assay is also useful for determining the IC₅₀ of a sodium channel blocking compound. The assay is based on the guanidine flux assay described by Reddy, N. L., et al., J. Med. Chem. (1998), 41(17):3298-302.

The guanidine influx assay is a radiotracer flux assay used to determine ion flux activity of sodium channels in a high-throughput microplate-based format. The assay uses ¹⁴C-guanidine hydrochloride in combination with various known sodium channel modulators to assay the potency of test agents. Potency is determined by an IC₅₀ calculation. Selectivity is determined by comparing potency of the compound for the channel of interest to its potency against other sodium channels (also called ‘selectivity profiling’).

Each of the test agents is assayed against cells that express the channels of interest. Voltage gated sodium channels are either TTX sensitive or insensitive. This property is useful when evaluating the activities of a channel of interest when it resides in a mixed population with other sodium channels. The following Table 1 summarizes cell lines useful in screening for a certain channel activity in the presence or absence of TTX.

TABLE 1

| CELL LINE | mRNA Expression | Functional Characterization |
|---|---|---|
| CHO-K1 (Chinese | Naᵥ1.4 expression has been | The 18- to 20-fold increase in |
| Hamster Ovary; | shown by RT-PCR | [¹⁴C] guanidine influx was |
| recommended | No other Na_{V} expression has | completely blocked using TTX. |
| host cell line) | been detected | (Na_{V}1.4 is a TTX sensitive |
| ATTC accession | | channel) |
| number CCL-61 | | |
| L6 (rat myoblast | Expression of Naᵥ1.4 and 1.5 | The 10- to 15-fold increase in |
| cell) ATTC | | [¹⁴C] guanidine influx was only |
| Number CRL-1458 | | partially blocked by TTX at 100 |
| | | nM (Naᵥ1.5 is TTX resistant) |
| SH-SY5Y (Human | Published Expression of | The 10- to 16-fold increase in |
| neuroblastoma) | Na_{V}1.9 and Na_{V}1.7 (Blum et | [¹⁴C] guanidine influx above |
| ATTC Number | al.) | background was partially blocked |
| CRL-2266 | | by TTX (Na_{V}1.9 is TTX resistant) |
| SK-N-BE2C (a | Expression of Na_{V}1.8 | Stimulation of BE2C cells with |
| human | | pyrethroids results in a 6-fold |
| neuroblastoma cell | | increase in [¹⁴C] guanidine influx |
| line ATCC Number | | above background. |
| CRL-2268) | | TTX partially blocked influx |
| | | (NaV1.8 is TTX resistant) |
| PC12 (rat | Expression of Naᵥ1.2 | The 8- to 12-fold increase in [¹⁴C] |
| pheochromocytoma) | expression | guanidine influx was completely |
| ATTC Number | | blocked using TTX. (Naᵥ1.2 is a |
| CRL-1721 | | TTX sensitive channel) |

It is also possible to employ recombinant cells expressing these sodium channels. Cloning and propagation of recombinant cells are known to those skilled in the art (see, for example, Klugbauer, N, et al., EMBO J. (1995), 14(6):1084-90; and Lossin, C., et al., Neuron (2002), 34:877-884).

Cells expressing the channel of interest are grown according to the supplier or in the case of a recombinant cell in the presence of selective growth media such as G418 (Gibco/Invitrogen). The cells are disassociated from the culture dishes with an enzymatic solution (1×) Trypsin/EDTA (Gibco/Invitrogen) and analyzed for density and viability using a haemocytometer (Neubauer). Disassociated cells are washed and resuspended in their culture media then plated into Scintiplates (Beckman Coulter Inc.) (approximately 100,000 cells/well) and incubated at 37° C./5% CO₂ for 20-24 hours. After an extensive wash with Low sodium HEPES-buffered saline solution (LNHBSS) (150 mM Choline Chloride, 20 nM HEPES (Sigma), 1 mM Calcium Chloride, 5 mM Potassium Chloride, 1 mM Magnesium Chloride, 10 mM Glucose) agents diluted with LNHBSS are added to each well. (Varying concentrations of test agent may be used). The activation/radiolabel mixture contains aconitine (Sigma) to increase the percentage of time that the sodium channels are open, and ¹⁴C-guanidine hydrochloride (ARC) to measure flux through the voltage-gated sodium channels.

After loading the cells with test agent and activation/radiolabel mixture, the Scintiplates are incubated at ambient temperature. Following the incubation, the Scintplates are extensively washed with LNHBSS supplemented with guanidine (Sigma). The Scintiplates are dried and then counted using a Wallac MicroBeta TriLux (Perkin-Elmer Life Sciences). The ability of the test agent to block sodium channel activity is determined by comparing the amount of ¹⁴C-guanidine present inside the cells expressing the different sodium channels. Based on this data, a variety of calculations, as set out elsewhere in this specification, may be used to determine whether a test agent is selective for a particular sodium channel.

The IC₅₀ value of a test agent for a specific sodium channel may be determined using the above general method. The IC₅₀ may be determined using a 3, 8, 10, 12 or 16 point curve in duplicate or triplicate with a starting concentration of 1, 5 or 10 μM diluted serially with a final concentration reaching the sub-nanomolar, nanomolar and low micromolar ranges. Typically the mid-point concentration of test agent is set at 1 μM, and sequential concentrations of half dilutions greater or smaller are applied (e.g. 0.5 μM; 5 μM and 0.25 μM; 10 μM and 0.125 μM; 20 μM etc.). The IC₅₀ curve is calculated using the 4 Parameter Logistic Model or Sigmoidal Dose-Response Model formula (fit=(A+((B−A)/(1+((C/×)̂D)))).

The fold selectivity, factor of selectivity or multiple of selectivity, is calculated by dividing the IC₅₀ value of the test sodium channel by the reference sodium channel, for example, Naᵥ1.5.

Representative compounds of the invention, when tested in the above assay using a known cell line that expresses a sodium channel, demonstrated an IC₅₀ (nM) activity level as set forth below in Table 2 wherein “A” refers to an IC₅₀ activity level of from 1 nM to 100 nM, “B” refers to an IC₅₀ activity level from 100 nM to 1 μM, “C” refers to an IC₅₀ activity level from 1 μM to 10 μM, and “D” refers to an IC₅₀ activity level from 10 μM to 100 μM. The Example numbers provided in Table 2 correspond to the Examples herein:

TABLE 2

| Ex. No. | Compound Name | IC₅₀ |
|---|---|---|
| 1 | 4′-bromo-5-methoxy-1′-[(2R)-tetrahydrofuran-2- | B |
| | ylmethyl]spiro[furo[3,2-b]pyridine-3,3′-indol]-2′(1′H)- | |
| | one | |
| 3 | 1′-(2,3-dihydro-1,4-benzodioxin-6-ylmethyl)-5- | A |
| | methoxyspiro[furo[3,2-b]pyridine-3,3′-indol]-2′(1′H)- | |
| | one | |
| 4 | 1′-[(2R)-tetrahydrofuran-2-ylmethyl]spiro[furo[2,3- | C |
| | c]pyridine-3,3′-indole]-2′,5(1′H,6H)-dione | |
| 6 | 2,3-dihydrospiro[furo[2,3-g][1,4]benzodioxine-8,3′- | B |
| | pyrrolo[2,3-b]pyridin]-2′(1′H)-one | |
| 7 | 1′-(pyridin-2-ylmethyl)-2,3-dihydrospiro[furo[2,3- | A |
| | g][1,4]benzodioxine-8,3′-pyrrolo[2,3-b]pyridin]- | |
| | 2′(1′H)-one | |
| 8 | 1′-{[3-(trifluoromethyl)pyridin-2-yl]methyl}-2,3- | B |
| | dihydrospiro[furo[2,3-g][1,4]benzodioxine-8,3′- | |
| | pyrrolo[2,3-b]pyridin]-2′(1′H)-one | |
| 10 | 2,3-dihydrospiro[furo[2,3-g][1,4]benzodioxine-8,3′- | C |
| | pyrrolo[3,2-b]pyridin]-2′(1′H)-one | |
| 11 | 1′-(pyridin-2-ylmethyl)-2,3-dihydrospiro[furo[2,3- | B |
| | g][1,4]benzodioxine-8,3′-pyrrolo[3,2-b]pyridin]- | |
| | 2′(1′H)-one | |
| 12 | 1′-{[3-(trifluoromethyl)pyridin-2-yl]methyl}-2,3- | B |
| | dihydrospiro[furo[2,3-g][1,4]benzodioxine-8,3′- | |
| | pyrrolo[3,2-b]pyridin]-2′(1′H)-one | |
| 13 | 5-methoxy-1′-{[3-(trifluoromethyl)pyridin-2- | B |
| | yl]methyl}spiro[furo[3,2-b]pyridine-3,3′-indol]-2′(1′H)- | |
| | one | |

### BIOLOGICAL EXAMPLE 2

### Electrophysiological Assay (In Vitro Assay)

Cells expressing the channel of interest are cultured in DMEM growth media (Gibco) with 0.5 mg/mL G418, +/−1% PSG, and 10% heat-inactivated fetal bovine serum at 37° C. and 5% CO₂. For electrophysiological recordings, cells are plated on 10 mm dishes.

Whole cell recordings are examined by established methods of whole cell voltage clamp (Bean et al., op. cit.) using an Axopatch 200B amplifier and Clampex software (Axon Instruments, Union City, Calif.). All experiments are performed at ambient temperature. Electrodes are fire-polished to resistances of 2-4 Mohms Voltage errors and capacitance artifacts are minimized by series resistance compensation and capacitance compensation, respectively. Data are acquired at 40 kHz and filtered at 5 kHz. The external (bath) solution consists of: NaCl (140 mM), KCl (5 mM), CaCl₂ (2 mM), MgCl₂ (1 mM), HEPES (10 mM) at pH 7.4. The internal (pipette) solution consists of (in mM): NaCl (5), CaCl₂ (0.1), MgCl₂ (2), CsCl (10), CsF (120), HEPES (10), EGTA (10), at pH 7.2.

To estimate the steady-state affinity of compounds for the resting and inactivated state of the channel (Kᵣ and Kᵢ, respectively), 12.5 ms test pulses to depolarizing voltages from −60 to +90 mV from a holding potential of −120 mV is used to construct current-voltage relationships (I-V curves). A voltage near the peak of the IV-curve (−30 to 0 mV) is used as the test pulse throughout the remainder of the experiment. Steady-state inactivation (availability) curves are then constructed by measuring the current activated during a 8.75 ms test pulse following 1 second conditioning pulses to potentials ranging from −120 to −10 mV.

The steady-state voltage-dependence of binding of a compound to a sodium channel is determined by measuring the blockage of the ionic current at two holding potentials. Binding to rested-state channels is determined by using a holding potential of −120 mV, so that maximal availability is achieved. Binding to inactivated-state channels is evaluated at a holding potential such that only 10% of the channels are available to open. The membrane potential is held at this voltage for at least 10 seconds so that drug binding can equilibrate.

The apparent dissociation constant at each voltage is calculated with the equation:

$$\% inhibition = \frac{[ Drug ]}{( [ Drug ] + {K}_{d} )}$$

where K_{d} is the dissociation constant (either Kᵣ or Kᵢ), and [Drug] is the concentration of the test compound.

Compounds of the invention may be tested in this model to determine the compounds' affinities for the inactivated state of the sodium channel of interest.

### BIOLOGICAL EXAMPLE 3

### Analgesia Induced by Sodium Channel Blockers

### Heat Induced Tail Flick Latency Test

In this test, the analgesia effect produced by administering a compound of the invention can be observed through heat-induced tail-flick in mice. The test includes a heat source consisting of a projector lamp with a light beam focused and directed to a point on the tail of a mouse being tested. The tail-flick latencies, which are assessed prior to drug treatment, and in response to a noxious heat stimulus, i.e., the response time from applying radiant heat on the dorsal surface of the tail to the occurrence of tail flick, are measured and recorded at 40, 80, 120, and 160 minutes.

For the first part of this study, 65 animals undergo assessment of baseline tail flick latency once a day over two consecutive days. These animals are then randomly assigned to one of the 11 different treatment groups including a vehicle control, a morphine control, and 9 compounds at 30 mg/Kg are administered intramuscularly. Following dose administration, the animals are closely monitored for signs of toxicity including tremor or seizure, hyperactivity, shallow, rapid or depressed breathing and failure to groom. The optimal incubation time for each compound is determined via regression analysis. The analgesic activity of the test compounds is expressed as a percentage of the maximum possible effect (% MPE) and is calculated using the following formula:

$$\% MPE \frac{Postdrug latency - Predrug latency}{Cut \text{-} off time ( 10 s ) - Predrug latency} \times 100 \%$$

where:

Postdrug latency=the latency time for each individual animal taken before the tail is removed (flicked) from the heat source after receiving drug.

Predrug latency=the latency time for each individual animal taken before the tail is flicked from the heat source prior to receiving drug.

Cut-off time (10 s)=is the maximum exposure to the heat source.

### Acute Pain (Formalin Test)

The formalin test is used as an animal model of acute pain. In the formalin test, animals are briefly habituated to the plexiglass test chamber on the day prior to experimental day for 20 minutes. On the test day, animals are randomly injected with the test articles. At 30 minutes after drug administration, 50 μL of 10% formalin is injected subcutaneously into the plantar surface of the left hind paw of the rats. Video data acquisition begins immediately after formalin administration, for duration of 90 minutes.

The images are captured using the Actimetrix Limelight software which stores files under the *.llii extension, and then converts it into the MPEG-4 coding. The videos are then analyzed using behaviour analysis software “The Observer 5.1”, (Version 5.0, Noldus Information Technology, Wageningen, The Netherlands). The video analysis is conducted by watching the animal behaviour and scoring each according to type, and defining the length of the behaviour (Dubuisson and Dennis, 1977). Scored behaviours include: (1) normal behaviour, (2) putting no weight on the paw, (3) raising the paw, (4) licking/biting or scratching the paw. Elevation, favoring, or excessive licking, biting and scratching of the injected paw indicate a pain response. Analgesic response or protection from compounds is indicated if both paws are resting on the floor with no obvious favoring, excessive licking, biting or scratching of the injected paw.

Analysis of the formalin test data is done according to two factors: (1) Percent Maximal Potential Inhibitory Effect (% MPIE) and (2) pain score. The % MPIEs is calculated by a series of steps, where the first is to sum the length of non-normal behaviours (behaviours 1, 2, 3) of each animal. A single value for the vehicle group is obtained by averaging all scores within the vehicle treatment group. The following calculation yields the MPIE value for each animal:

MPIE(%)=100−[(treatment sum/average vehicle value)×100%]

The pain score is calculated from a weighted scale as described above. The duration of the behaviour is multiplied by the weight (rating of the severity of the response), and divided by the total length of observation to determine a pain rating for each animal. The calculation is represented by the following formula:

Pain rating=[0(To)+1(T1)+2(T2)+3(T3)]/(To+T1+T2+T3)

### CFA Induced Chronic Inflammatory Pain

In this test, tactile allodynia is assessed with calibrated von Frey filaments. Following a full week of acclimatization to the vivarium facility, 150 μL of the “Complete Freund's Adjuvant” (CFA) emulsion (CFA suspended in an oil/saline (1:1) emulsion at a concentration of 0.5 mg/mL) is injected subcutaneously into the plantar surface of the left hind paw of rats under light isoflurane anaesthesia. Animals are allowed to recover from the anaesthesia and the baseline thermal and mechanical nociceptive thresholds of all animals are assessed one week after the administration of CFA. All animals are habituated to the experimental equipment for 20 minutes on the day prior to the start of the experiment. The test and control articles are administrated to the animals, and the nociceptive thresholds measured at defined time points after drug administration to determine the analgesic responses to each of the six available treatments. The time points used are previously determined to show the highest analgesic effect for each test compound.

Thermal nociceptive thresholds of the animals are assessed using the Hargreaves test. Animals are placed in a Plexiglas enclosure set on top of an elevated glass platform with heating units. The glass platform is thermostatically controlled at a temperature of approximately 30° C. for all test trials. Animals are allowed to accommodate for 20 minutes following placement into the enclosure until all exploration behaviour ceases. The Model 226 Plantar/Tail Stimulator Analgesia Meter (IITC, Woodland Hills, Calif.) is used to apply a radiant heat beam from underneath the glass platform to the plantar surface of the hind paws. During all test trials, the idle intensity and active intensity of the heat source are set at 1 and 45 respectively, and a cut off time of 20 seconds is employed to prevent tissue damage.

The response thresholds of animals to tactile stimuli are measured using the Model 2290 Electrovonfrey anesthesiometer (IITC Life Science, Woodland Hills, Calif.) following the Hargreaves test. Animals are placed in an elevated Plexiglas enclosure set on a mire mesh surface. After 10 minutes of accommodation, pre-calibrated Von Frey hairs are applied perpendicularly to the plantar surface of both paws of the animals in an ascending order starting from the 0.1 g hair, with sufficient force to cause slight buckling of the hair against the paw. Testing continues until the hair with the lowest force to induce a rapid flicking of the paw is determined or when the cut off force of approximately 20 g is reached. This cut off force is used because it represent approximately 10% of the animals' body weight and it serves to prevent raising of the entire limb due to the use of stiffer hairs, which would change the nature of the stimulus.

### Postoperative Models of Nociception

In this model, the hypealgesia caused by an intra-planar incision in the paw is measured by applying increased tactile stimuli to the paw until the animal withdraws its paw from the applied stimuli. While animals are anaesthetized under 3.5% isofluorane, which is delivered via a nose cone, a 1 cm longitudinal incision is made using a number 10 scalpel blade in the plantar aspect of the left hind paw through the skin and fascia, starting 0.5 cm from the proximal edge of the heel and extending towards the toes. Following the incision, the skin is apposed using 2, 3-0 sterilized silk sutures. The injured site is covered with Polysporin and Betadine. Animals are returned to their home cage for overnight recovery.

The withdrawal thresholds of animals to tactile stimuli for both operated (ipsilateral) and unoperated (contralateral) paws can be measured using the Model 2290 Electrovonfrey anesthesiometer (IITC Life Science, Woodland Hills, Calif.). Animals are placed in an elevated Plexiglas enclosure set on a mire mesh surface. After at least 10 minutes of acclimatization, pre-calibrated Von Frey hairs are applied perpendicularly to the plantar surface of both paws of the animals in an ascending order starting from the 10 g hair, with sufficient force to cause slight buckling of the hair against the paw. Testing continues until the hair with the lowest force to induce a rapid flicking of the paw is determined or when the cut off force of approximately 20 g is reached. This cut off force is used because it represent approximately 10% of the animals' body weight and it serves to prevent raising of the entire limb due to the use of stiffer hairs, which would change the nature of the stimulus.

### Neuropathic Pain Model; Chronic Constriction Injury

Briefly, an approximately 3 cm incision is made through the skin and the fascia at the mid thigh level of the animals' left hind leg using a no. 10 scalpel blade. The left sciatic nerve is exposed via blunt dissection through the biceps femoris with care to minimize haemorrhagia. Four loose ligatures are tied along the sciatic nerve using 4-0 non-degradable sterilized silk sutures at intervals of 1 to 2 mm apart. The tension of the loose ligatures is tight enough to induce slight constriction of the sciatic nerve when viewed under a dissection microscope at a magnification of 4 fold. In the sham-operated animal, the left sciatic nerve is exposed without further manipulation. Antibacterial ointment is applied directly into the wound, and the muscle is closed using sterilized sutures. Betadine is applied onto the muscle and its surroundings, followed by skin closure with surgical clips.

The response thresholds of animals to tactile stimuli are measured using the Model 2290 Electrovonfrey anesthesiometer (IITC Life Science, Woodland Hills, Calif.). Animals are placed in an elevated Plexiglas enclosure set on a mire mesh surface. After 10 minutes of accommodation, pre-calibrated Von Frey hairs are applied perpendicularly to the plantar surface of both paws of the animals in an ascending order starting from the 0.1 g hair, with sufficient force to cause slight buckling of the hair against the paw. Testing continues until the hair with the lowest force to induce a rapid flicking of the paw is determined or when the cut off force of approximately 20 g is reached. This cut off force is used because it represents approximately 10% of the animals' body weight and it serves to prevent raising of the entire limb due to the use of stiffer hairs, which would change the nature of the stimulus.

Thermal nociceptive thresholds of the animals are assessed using the Hargreaves test. Following the measurement of tactile thresholds, animals are placed in a Plexiglass enclosure set on top of an elevated glass platform with heating units. The glass platform is thermostatically controlled at a temperature of approximately 24 to 26° C. for all test trials. Animals are allowed to accommodate for 10 minutes following placement into the enclosure until all exploration behaviour ceases. The Model 226 Plantar/Tail Stimulator Analgesia Meter (IITC, Woodland Hills, Calif.) is used to apply a radiant heat beam from underneath the glass platform to the plantar surface of the hind paws. During all test trials, the idle intensity and active intensity of the heat source are set at 1 and 55 respectively, and a cut off time of 20 seconds is used to prevent tissue damage.

### Neuropathic Pain Model: Spinal Nerve Ligation

The spinal nerve ligation (SNL) neuropathic pain model is used as an animal (i.e. rat) model of neuropathic pain. In the SNL test, the lumbar roots of spinal nerves L5 and L6 are tightly ligated to cause nerve injury, which results in the development of mechanical hyperalgesia, mechanical allodynia and thermal hypersensitivity. The surgery is performed two weeks before the test day in order for the pain state to fully develop in the animals. Several spinal nerve ligation variations are used to characterize the analgesic properties of a compound of the invention.
(1) Ligation of the L5 spinal nerve;
(2) Ligation of the L5 and L6 spinal nerves;
(3) Ligation and transection of the L5 spinal nerve;
(4) Ligation and transection of the L5 and L6 spinal nerves; or
(5) Mild irritation of the L4 spinal nerve in combination with any one of the above (1)-(4).

While the animals are anaesthetized under 3.5% isofluorane delivered via a nose cone, an approximately 2.5 cm longitudinal incision is made using a number 10 scalpel blade in the skin just lateral to the dorsal midline, using the level of the posterior iliac crests as the midpoint of the incision. Following the incision, the isoflourane is readjusted to maintenance levels (1.5%-2.5%). At mid-sacral region, an incision is made with the scalpel blade, sliding the blade along the side of the vertebral column (in the saggital plane) until the blade hits the sacrum. Scissors tips are introduced through the incision and the muscle and ligaments are removed from the spine to expose 2-3 cm of the vertebral column. The muscle and fascia are cleared from the spinal vertebra in order to locate the point where the nerve exits from the vertebra. A small glass hook is placed medial to the spinal nerves and the spinal nerves are gently elevated from the surrounding tissues. Once the spinal nerves have been isolated, a small length of non-degradable 6-0 sterilized silk thread is wound twice around the ball at the tip of the glass hook and passed back under the nerve. The spinal nerves are then firmly ligated by tying a knot, ensuring that the nerve bulges on both sides of the ligature. The procedure may be repeated as needed. In some animals, the L4 spinal nerve may be lightly rubbed (up to 20 times) with the small glass hook to maximize the development of neuropathic pain. Antibacterial ointment is applied directly into the incision, and the muscle is closed using sterilized sutures. Betadine is applied onto the muscle and its surroundings, followed by skin closure with surgical staples or sterile non-absorable monofilament 5-0 nylon sutures.

The analgesic effect produced by topical administration of a compound of the invention to the animals can then be observed by measuring the paw withdrawal threshold of animals to mechanical tactile stimuli. These may be measured using either the mechanical allodynia procedure or the mechanical hyperalgesia procedure as described below. After establishment of the appropriate baseline measurements by either method, topical formulation of a compound of the invention is applied on the ipsilateral ankle and foot. The animals are then placed in plastic tunnels for 15 minutes to prevent them from licking the treated area and removing the compound. Animals are placed in the acrylic enclosure for 15 minutes before testing the ipsilateral paw by either of the methods described below, and the responses are recorded at 0.5, 1.0 and 2.0 hour post treatment.

A. Mechanical Allodynia Method

The pain threshold of animals to mechanical alloydnia for both operated and control animals can be measured approximately 14 days post-surgery using manual calibrated von Frey filaments as follows. Animals are placed in an elevated plexiglass enclosure set on a mire mesh surface. Animals are allowed to acclimate for 20-30 minutes. Pre-calibrated Von Frey hairs are applied perpendicularly to the plantar surface of the ipsilateral paw of the animals starting from the 2.0 g hair, with sufficient force to cause slight buckling of the hair against the paw to establish the baseline measurements. Stimuli are presented in a consecutive manner, either in an ascending or descending order until the first change in response is noted, after which four additional responses are recorded for a total of six responses. The six responses measured in grams are entered into a formula as described by Chaplan, S. R. et al., J. Neurosci. Methods, 1994 July; 53(1):55-63, and a 50% withdrawal threshold is calculated. This constitutes the mechanical allodynia value.

B. Mechanical Hyperalgesia Method

The response thresholds of animals to tactile stimuli were measured using the Model 2290 Electrovonfrey anesthesiometer (IITC Life Science, Woodland Hills, Calif.). Animals were placed in an elevated Plexiglas enclosure set on a wire mesh surface. After 15 minutes of accommodation in this enclosure, a von Frey hair was applied perpendicularly to the plantar surface of the ipsilateral hind paws of the animals, with sufficient force, measured in grams, to elicit a crisp response of the paw. The response indicated a withdrawal from the painful stimulus and constituted the efficacy endpoint. The data were expressed as percent change from baseline threshold measured in grams.

### BIOLOGICAL EXAMPLE 4

### Aconitine Induced Arrhythmia Test

The antiarrhythmic activity of compounds of the invention is demonstrated by the following test. Arrhythmia is provoked by intravenous administration of aconitine (2.0 μg/Kg) dissolved in physiological saline solution. Test compounds of the invention are intravenously administered 5 minutes after the administration of aconitine. Evaluation of the anti-arrhythmic activity is conducted by measuring the time from the aconitine administration to the occurrence of extrasystole (ES) and the time from the aconitine administration to the occurrence of ventricular tachycardia (VT).

In rats under isoflurane anaesthesia (¼ to ⅓ of 2%), a tracheotomy is performed by first creating an incision in the neck area, then isolating the trachea and making a 2 mm incision to insert tracheal tube 2 cm into the trachea such that the opening of the tube is positioned just on top of the mouth. The tubing is secured with sutures and attached to a ventilator for the duration of the experiment.

Incisions (2.5 cm) are then made into the femoral areas and using a blunt dissection probe, the femoral vessels are isolated. Both femoral veins are cannulated, one for pentobarbital anaesthetic maintenance (0.02-0.05 mL) and one for the infusion and injection of drug and vehicle. The femoral artery is cannulated with the blood pressure gel catheter of the transmitter.

The ECG leads are attached to the thoracic muscle in the Lead II position (upper right/above heart—white lead and lower left/below heart—red lead). The leads are secured with sutures.

All surgical areas are covered with gauze moistened with 0.9% saline. Saline (1-1.5 mL of a 0.9% solution) is supplied to moisten the areas post-surgery. The animals' ECG and ventillation are allowed to equilibrate for at least 30 minutes.

The arrhythmia is induced with a 2 μg/Kg/min aconitine infusion for 5 minutes. During this time the ECG is recorded and continuously monitored. Compounds of the present invention can be tested in these assays to determine their effectiveness in treating arrhythmia.

### BIOLOGICAL EXAMPLE 5

### Ischemia Induced Arrhythmia Test

Rodent models of ventricular arrhythmias, in both acute cardioversion and prevention paradigms have been employed in testing potential therapeutics for both atrial and ventricular arrhythmias in humans. Cardiac ischemia leading to myocardial infarction is a common cause of morbidity and mortality. The ability of a compound to prevent ischemia-induced ventricular tachycardia and fibrillation is an accepted model for determining the efficacy of a compound in a clinical setting for both atrial and ventricular tachycardia and fibrillation.

Anaesthesia is first induced by pentobarbital (i.p.), and maintained by an i.v. bolus infusion. Male SD rats have their trachea cannulated for artificial ventilation with room air at a stroke volume of 10 mL/Kg, 60 strokes/minute. The right femoral artery and vein are cannulated with PE50 tubing for mean arterial blood pressure (MAP) recording and intravenous administration of compounds, respectively.

The chest is opened between the 4^{th} and 5^{th} ribs to create a 1.5 cm opening such that the heart is visible. Each rat is placed on a notched platform and metal restraints are hooked onto the rib cage opening the chest cavity. A suture needle is used to penetrate the ventricle just under the lifted atrium and exited the ventricle in a downward diagonal direction so that a >30% to <50% occlusion zone (OZ) would be obtained. The exit position is ˜0.5 cm below where the aorta connects to the left ventricle. The suture is tightened such that a loose loop (occluder) is formed around a branch of the artery. The chest is then closed with the end of the occluder accessible outside of the chest.

Electrodes are placed in the Lead II position (right atrium to apex) for ECG measurement as follows: one electrode inserted into the right forepaw and the other electrode inserted into the left hind paw.

The body temperature, MAP, ECG, and heart rate are constantly recorded throughout the experiment. Once the critical parameters has stabilized, a 1-2 minute recording is taken to establish the baseline values. Infusion of a compound of the invention or control substance is initiated once baseline values are established. After a 5-minute infusion of compound or control, the suture is pulled tight to ligate the LCA and create ischemia in the left ventricle. The critical parameters are recorded continuously for 20 minutes after ligation, unless the MAP reached the critical level of 20-30 mmHg for at least 3 minutes, in which case the recording is stopped because the animal would be declared deceased and is then sacrificed. The ability of compounds of the invention to prevent arrhythmias and sustain near-normal MAP and HR is scored and compared to control.

### BIOLOGICAL EXAMPLE 6

### In Vivo Assay for Benign Prostate Hyperplasia (BPH)

The effectiveness of the compounds of the present invention for treating BPH can be demonstrated by the following in vivo assay.

Dogs are dosed orally with compounds of the present invention at oral doses of between 0 mg/Kg and 100 mg/Kg for a period of 4 weeks. A control group receives placebo. The animals are sacrificed and the prostate glands dissected out, dabbed dry and then weighed.

### BIOLOGICAL EXAMPLE 7

### In Vivo Assay for Antihypercholesterlemia Efficacy and Antiatherosclerotic Efficacy

Dogs have cardiovascular systems similar to that of humans, making them ideal for studying the effects of medicinal compounds designed to treat cardiovascular disorders.

Dogs are dosed orally at a range of 0 mg/Kg to 100 mg/Kg daily with compounds of the present invention for a period of 2-4 weeks. After 2 and 4 weeks the animals are bled and their serum collected for total cholesterol analysis and compared to the animals dosed with vehicle alone (0 mg/Kg).

The measurement of cholesterol is one of the most common tests performed in the clinical laboratory setting. Simple fluorometric methods for the sensitive quantitation of total cholesterol in plasma or serum are commonly used. In one assay, cholesteryl esters in the sample are first hydrolyzed by cholesterol esterase. All cholesterol, whether previously esterified or existing free in the circulation, is then oxidized by cholesterol oxidase to the corresponding ketone and hydrogen peroxide. ADHP (10-acetyl-3,7-dihydroxyphenoxazine) is utilized as a highly sensitive and stable probe for hydrogen peroxide. Horseradish peroxidase catalyzes the reaction of ADHP with hydrogen peroxide to yield the highly fluorescent product resorufin, which can be monitored using excitation wavelengths of 565-580 nm and emission wavelengths of 585-595 nm.

### BIOLOGICAL EXAMPLE 8

### In Vivo Assay for Treatment of Pruritis

The compounds of the invention can be evaluated for their activity as antipruritic agents by in vivo test using rodent models. One established model for peripherally elicited pruritus is through the injection of serotonin into the rostral back area (neck) in hairless rats. Prior to serotonin injections (e.g., 2 mg/mL, 50 μL), a dose of a compound of the present invention can be applied systemically through oral, intravenous or intraperitoneal routes or topically to a circular area fixed diameter (e.g. 18 mm). Following dosing, the serotonin injections are given in the area of the topical dosing. After serotonin injection the animal behaviour is monitored by video recording for 20 min-1.5 h, and the number of scratches in this time compared to vehicle treated animals. Thus, application of a compound of the current invention could suppress serotonin-induced scratching in rats.

### BIOLOGICAL EXAMPLE 9

### Cytochrome P450 (CYP450) Inhibition Assay

CYP450 (CYP) is a designation for a superfamily of enzymes. Each family consists of one or more subfamilies and each subfamily contains one or more specific CYP isoforms. The Cytochrome P450 (CYP450) Inhibition Assay is a fluorescence-based assay using a cytochrome CYP isozyme for screening of compounds of the invention to determine the level of CYP inhibition by a specific compound. The assay is based on the CYP inhibition kit described by Vivid CYP450 Screening Kit Protocol, 2005, Invitrogen Corporation (Invitrogen Corporation, 1600 Faraday Avenue, Carlsbad, Calif. 92008, USA).

This assay is designed to assess compounds by quantifying the inhibition of the predominant human CYP isozymes involved in hepatic drug metabolism. It is based on the principle derived from the testing of many pharmacologically active compounds for their ability to serve as substrates and inhibitors for the major Drug Metabolizing Enzymes, primarily CYPs, or for their interference with the metabolism of existing drugs. The standard method for evaluating specific CYP isozyme inhibition is to determine the conversion rate of a probe substrate into its metabolite, in the presence and absence of the potential inhibitor. Quantification of the metabolite is achieved by HPLC or by using a probe substrate (Table 3) that is metabolized into a fluorescent product (fluorescent assay).

Four CYP isozymes were investigated: CYP3A4, 2C9, 2C19 and 2D6. In particular, CYP3A4 is shown to be one of the most important isozyme involved in the metabolism of drugs in the body (see http://medicine.iupui.edu/flockhart/table.htm). A drug that inhibits a specific CYP isozyme may decrease the metabolism of the drug and therefore increase serum concentrations of drugs that are substrates for that isoenzyme.

This assay can be used for single concentration screening or for IC₅₀ determination. In a single concentration screening assay, the final assay concentration of the test compound is 10 μM. In an IC₅₀ determination assay, IC₅₀ may be determined using a 3, 6, or 12 point curve in triplicate with a chosen starting concentration diluted serially.

### Preparation Stage:

In the Preparation Stage, the test compounds, controls (acetonitrile (ACN) or Dimethyl sulfoxide (DMSO) and No Baculosomes), and known inhibitors (Table 4) were diluted to 10% ACN or DMSO in water at appropriate concentrations. The Premix and Substrate Mix solutions were also prepared per kit instructions. The Premix consisted of P450 Baculosomes, regeneration system (RS), and Vivid® CYP450 reaction buffer. The Substrate Mix consisted of Vivid® substrate, NADP+ and Vivid® CYP450 reaction buffer.

### Assay Stage:

In the Assay Stage, 30 μL water was added to each well of a 96-well assay plate. Then 10 μL of the 10% ACN or DMSO in water stocks of the test compounds, negative controls, or known inhibitors were added to designated wells according to the assay plate layout. The third step was to add 50 μL of the Premix solution to each working well (except for No Baculosomes control wells, 50 μL buffer was added instead). The assay plate was then pre-warmed at ambient temperature in the dark for 20 minutes. When pre-warming was completed, 10 μL of the Substrate Mix solution was added to each working well (including the No Baculosomes control wells). This resulted in a final 1% ACN or DMSO concentration. The assay plate was immediately placed in a PolarStar plate reader to read initial fluorescence. The assay plate was again incubated at ambient temperature in the dark for 20, 30, or 60 minutes, depending on the reaction time of the isozyme (Table 5). 10 μL of the stop reagent was added to each working well and final fluorescence was read.

TABLE 3
CYP450 ISOZYMES (CYP) AND SUBSTRATES USED

| CYP | Substrate Acronym | Structure Name |
|---|---|---|
| 3A4 | BOMCC | 7-(benzyloxymethoxy)-3-cyanocoumarin |
| 2C19 | EOMCC | 7-(ethoxymethoxy)-3-cyanocoumarin |
| 2C9 | BOMF | (benzyloxymethoxy)fluorescein |
| 2D6 | MOBFC | 7-(4-methoxybenzyloxy)-4- |
| | | trifluoromethylcoumarin |

TABLE 4
CYP450 ISOZYME INHIBITORS

| Iso- | | Final Assay | | |
|---|---|---|---|---|
| zyme | Inhibitor | Concentration | % Inhibition | IC₅₀ (nM) |
| 3A4 | Ketoconazole | 0.1 μM | 50 +/− 10% | 88 +/− 30 nM |
| 2C9 | Sulfaphen- | 0.420 μM | 50 +/− 15% | 345 +/− 20 nM |
| | azole | | | |
| 2C19 | Ketoconazole | 7.62 μM | 65 +/− 10% | 3132 +/− 680 nM |
| 2D6 | Quinidine | 0.0137 μM | 55 +/− 15% | 15 +/− 5 nM |

TABLE 5
ISOZYME REACTION TIME AND STOP REAGENT

| Isozyme | Reaction Time (min) | Concentration of Stop Reagent |
|---|---|---|
| 3A4 | 20 | 10 μM Ketoconazole |
| 2C19 | 20 | 30 μM Ketoconazole |
| 2C9 | 30 | 10 μM Sulfaphenazole |
| 2D6 | 60 | 1 μM Quinidine |

TABLE 6
TERMINOLOGY

| | |
|---|---|
| Regener- | 100x consists of 333 mM Glucose-6-phosphate and 40 |
| ation System | U/mL Glucose-6-phosphate dehydrogenase in 100 mM |
| (RS) | Potassium Phosphate Buffer (pH 8.0). |
| Baculosomes | Microsomes prepared from insect cells that were infected |
| (Bac) | with baculovirus containing the cDNAs for human CYP |
| | isozyme (1 μM specific P450 content) and rabbit NADPH |
| | reductase. |
| NADP⁺ | Nicotinamide adenine dinucleotide phosphate at 10 μM in |
| | potassium phosphate buffer (100 mM, pH 8.0). Conversion |
| | of NADP+ into NADPH by the regeneration system is |
| | required to start the CYP450 reaction. |
| DMSO | Dimethyl sulfoxide |
| Reaction | Contains 100 or 200 mM potassium phosphate buffer. |
| Buffer | |
| Pre-Mix | Contains reaction buffer, RS, Bac. Prepare separately for |
| | each isozyme. |
| Substrate | Contains reaction buffer, substrate (BOMCC, EOMCC, |
| Mix | BOMF, or MOBFC), and NADP+. Prepare separately for |
| | each isozyme. |
| RFU | Relative fluorescence unit |

### Data Analysis

The difference between the initial and final fluorescence readings was used to calculate percent inhibition. The ACN or DMSO readings represented 0% inhibition and the No Baculosomes readings represented 100% inhibition. Percent inhibition by the compound or known inhibitor was calculated based on comparison with the solvent (ACN or DMSO) control and the No Baculosomes control. To minimize the any fluorescence compound or background effect, the relative fluorescence unit (RFU) initial was subtracted from the RFU final.

Determine the % inhibition for each compound or control for each CYP450 isozyme:

$$\% Inhibition = \frac{\begin{matrix}Compound ( RFU final \text{-} i nitial ) - \\ DMSO control ( RFU final \text{-} initial )\end{matrix} }{\begin{matrix}NoBac ( RFU final \text{-} initial ) - \\ DMSO control ( RFU final \text{-} initial )\end{matrix}} \times 100$$

Representative compounds of the invention, when tested in the above assay demonstrated percent inhibition of the CYP3A4 isozyme as set forth below in Table 7 wherein “A” refers to percent inhibition of less than 50% at 10 μM and “B” refers to percent inhibition of greater than 50% at 10 μM. The Example numbers provided in Table 7 correspond to the Examples herein:

TABLE 7

| | | % Inhibition |
|---|---|---|
| Ex. No. | Compound Name | of CYP3A4 |
| 1 | 4′-bromo-5-methoxy-1′-[(2R)-tetrahydrofuran-2- | A |
| | ylmethyl]spiro[furo[3,2-b]pyridine-3,3′-indol]-2′(1′H)-one | |
| 4 | 1′-[(2R)-tetrahydrofuran-2-ylmethyl]spiro[furo[2,3-c]pyridine- | A |
| | 3,3′-indole]-2′,5(1′H,6H)-dione | |
| 6 | 2,3-dihydrospiro[furo[2,3-g][1,4]benzodioxine-8,3′-pyrrolo[2,3- | A |
| | b]pyridin]-2′(1′H)-one | |
| 7 | 1′-(pyridin-2-ylmethyl)-2,3-dihydrospiro[furo[2,3- | A |
| | g][1,4]benzodioxine-8,3′-pyrrolo[2,3-b]pyridin]-2′(1′H)- | |
| | one | |
| 8 | 1′-{[3-(trifluoromethyl)pyridin-2-yl]methyl}-2,3- | A |
| | dihydrospiro[furo[2,3-g][1,4]benzodioxine-8,3′- | |
| | pyrrolo[2,3-b]pyridin]-2′(1′H)-one | |
| 10 | 2,3-dihydrospiro[furo[2,3-g][1,4]benzodioxine-8,3′-pyrrolo[3,2- | A |
| | b]pyridin]-2′(1′H)-one | |
| 11 | 1′-(pyridin-2-ylmethyl)-2,3-dihydrospiro[furo[2,3- | A |
| | g][1,4]benzodioxine-8,3′-pyrrolo[3,2-b]pyridin]-2′(1′H)- | |
| | one | |
| 12 | 1′-{[3-(trifluoromethyl)pyridin-2-yl]methyl}-2,3- | A |
| | dihydrospiro[furo[2,3-g][1,4]benzodioxine-8,3′- | |
| | pyrrolo[3,2-b]pyridin]-2′(1′H)-one | |
| 13 | 5-methoxy-1′-{[3-(trifluoromethyl)pyridin-2- | A |
| | yl]methyl}spiro[furo[3,2-b]pyridine-3,3′-indol]-2′(1′H)-one | |

All of the U.S. patents, U.S. patent application publications, U.S. patent applications, foreign patents, foreign patent applications and non-patent publications referred to in this specification are incorporated herein by reference in their entireties.

Although the foregoing invention has been described in some detail to facilitate understanding, it will be apparent that certain changes and modifications may be practiced within the scope of the appended claims. Accordingly, the described embodiments are to be considered as illustrative and not restrictive, and the invention is not to be limited to the details given herein, but may be modified within the scope and equivalents of the appended claims.

What is claimed is:

1. A compound of formula (I): 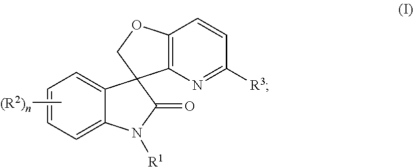
wherein:
n is 1 or 2;
R¹ is [3-(trifluoromethyl)pyridin-2-yl]methyl, tetrahydrofuran-2-ylmethyl, (2R)-tetrahydrofuran-2-ylmethyl, (2S)-tetrahydrofuran-2-ylmethyl or 2,3-dihydro-1,4-benzodioxin-6-ylmethyl;
each R² is independently selected from hydrogen or halo; and
R³ is methoxy, ethoxy or halo;
as a stereoisomer, enantiomer, tautomer thereof or mixtures thereof;
or a pharmaceutically acceptable salt, solvate or prodrug thereof.

2. The compound of claim 1 selected from:
4′-bromo-5-methoxy-1′-[(2R)-tetrahydrofuran-2-ylmethyl]spiro[furo[3,2-b]pyridine-3,3′-indol]-2′(1′H)-one;
5-methoxy-1′-[(2R)-tetrahydrofuran-2-ylmethyl]spiro[furo[3,2-b]pyridine-3,3′-indol]-2′(1′H)-one;
1′-(2,3-dihydro-1,4-benzodioxin-6-ylmethyl)-5-methoxyspiro[furo[3,2-b]pyridine-3,3′-indol]-2′(1′H)-one; or
5-methoxy-1′-{[3-(trifluoromethyl)pyridin-2-yl]methyl}spiro[furo[3,2-b]pyridine-3,3′-indol]-2′(1H)-one.

3. A compound of formula (II): 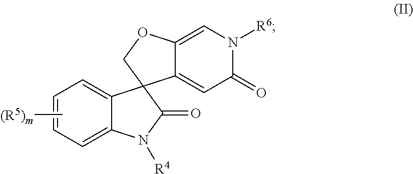
wherein:
m is 1 or 2;
R⁴ is [3-(trifluoromethyl)pyridin-2-yl]methyl, tetrahydrofuran-2-ylmethyl, (2R)-tetrahydrofuran-2-ylmethyl or (2S)-tetrahydrofuran-2-ylmethyl;
each R⁵ is independently selected from hydrogen or halo; and
R⁶ is hydrogen or alkyl;
as a stereoisomer, enantiomer, tautomer thereof or mixtures thereof;
or a pharmaceutically acceptable salt, solvate or prodrug thereof.

4. The compound of claim 3 which is 1′-[(2R)-tetrahydrofuran-2-ylmethyl]spiro[furo[2,3-c]pyridine-3,3′-indole]-2′,5(1′H,6H)-dione.

5. A compound of formula (III): 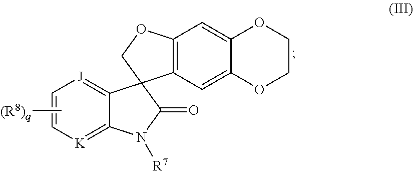
wherein:
q is 1 or 2;
one of J and K is —N═ and the other is —C(R⁸)═;
R⁷ is hydrogen, diphenylmethyl, pyridin-2-ylmethyl or [3-(trifluoromethyl)pyridin-2-yl]methyl; and
each R⁸ is independently selected from hydrogen or halo;
as a stereoisomer, enantiomer, tautomer thereof or mixtures thereof;
or a pharmaceutically acceptable salt, solvate or prodrug thereof.

6. The compound of claim 5 wherein J is —N═ and K is —C(R⁸)═.

7. The compound of claim 6 selected from:
1′-(diphenylmethyl)-2,3-dihydrospiro[furo[2,3-g][1,4]benzodioxine-8,3′-pyrrolo[3,2-b]pyridin]-2′(1′H)-one;
2,3-dihydrospiro[furo[2,3-g][1,4]benzodioxine-8,3′-pyrrolo[3,2-b]pyridin]-2′(1′H)-one;
1′-(pyridin-2-ylmethyl)-2,3-dihydrospiro[furo[2,3-g][1,4]benzodioxine-8,3′-pyrrolo[3,2-b]pyridin]-2′(1′H)-one; or
1′-{[3-(trifluoromethyl)pyridin-2-yl]methyl}-2,3-dihydrospiro[furo[2,3-g][1,4]benzodioxine-8,3′-pyrrolo[3,2-b]pyridin]-2′(1′H)-one.

8. The compound of claim 5 wherein J is —C(R⁸)═ and K is —N═.

9. The compound of claim 8 selected from:
1′-(diphenylmethyl)-2,3-dihydrospiro[furo[2,3-g][1,4]benzodioxine-8,3′-pyrrolo[2,3-b]pyridin]-2′(1H)-one;
2,3-dihydrospiro[furo[2,3-g][1,4]benzodioxine-8,3′-pyrrolo[2,3-b]pyridin]-2′(1′H)-one;
1′-(pyridin-2-ylmethyl)-2,3-dihydrospiro[furo[2,3-g][1,4]benzodioxine-8,3′-pyrrolo[2,3-b]pyridin]-2′(1′H)-one; or
1′-{[3-(trifluoromethyl)pyridin-2-yl]methyl}-2,3-dihydrospiro[furo[2,3-g][1,4]benzodioxine-8,3′-pyrrolo[2,3-b]pyridin]-2′(1′H)-one.

10. A pharmaceutical composition comprising a pharmaceutically acceptable excipient and a compound of claim 1, claim 3 or claim 5, as a stereoisomer, enantiomer, tautomer thereof or mixtures thereof; or a pharmaceutically acceptable salt, solvate or prodrug thereof.

11. A method of treating, preventing or ameliorating a disease or a condition in a mammal selected from the group consisting of pain, depression, cardiovascular diseases, respiratory diseases, and psychiatric diseases, and combinations thereof, wherein the method comprises administering to the mammal in need thereof a therapeutically effective amount of a compound of claim 1, claim 3 or claim 5, as a stereoisomer, enantiomer, tautomer thereof or mixtures thereof; or a pharmaceutically acceptable salt, solvate or prodrug thereof.

12. The method of claim 11, wherein said disease or condition is selected from the group consisting of neuropathic pain, inflammatory pain, visceral pain, cancer pain, chemotherapy pain, trauma pain, surgical pain, post-surgical pain, childbirth pain, labor pain, neurogenic bladder, ulcerative colitis, chronic pain, dental pain, persistent pain, peripherally mediated pain, centrally mediated pain, chronic headache, migraine headache, sinus headache, tension headache, phantom limb pain, peripheral nerve injury, and combinations thereof.

13. The method of claim 11, wherein said disease or condition is selected from the group consisting of pain associated with HIV, HIV treatment induced neuropathy, trigeminal neuralgia, post-herpetic neuralgia, eudynia, heat sensitivity, tosarcoidosis, irritable bowel syndrome, Crohns disease, pain associated with multiple sclerosis (MS), amyotrophic lateral sclerosis (ALS), diabetic neuropathy, peripheral neuropathy, arthritic, rheumatoid arthritis, osteoarthritis, atherosclerosis, paroxysmal dystonia, myasthenia syndromes, myotonia, malignant hyperthermia, cystic fibrosis, pseudoaldosteronism, rhabdomyolysis, hypothyroidism, bipolar depression, anxiety, schizophrenia, sodium channel toxin related illnesses, familial erythermalgia, primary erythermalgia, familial rectal pain, cancer, epilepsy, partial and general tonic seizures, restless leg syndrome, arrhythmias, fibromyalgia, neuroprotection under ischaemic conditions caused by stroke or neural trauma, tachy-arrhythmias, atrial fibrillation and ventricular fibrillation.

14. A method of treating pain in a mammal by the inhibition of ion flux through a voltage-dependent sodium channel in the mammal, wherein the method comprises administering to the mammal in need thereof a therapeutically effective amount of a compound of claim 1, claim 3 or claim 5, as a stereoisomer, enantiomer, tautomer thereof or mixtures thereof; or a pharmaceutically acceptable salt, solvate or prodrug thereof.

15. A method of decreasing ion flux through a voltage-dependent sodium channel in a cell in a mammal, wherein the method comprises contacting the cell with a compound of claim 1, claim 3 or claim 5, as a stereoisomer, enantiomer, tautomer thereof or mixtures thereof; or a pharmaceutically acceptable salt, solvate or prodrug thereof.

16. A method of treating hypercholesterolemia in a mammal, wherein the method comprises administering to the mammal in need thereof a therapeutically effective amount of a compound of claim 1, claim 3 or claim 5, as a stereoisomer, enantiomer, tautomer thereof or mixtures thereof; or a pharmaceutically acceptable salt, solvate or prodrug thereof.

17. A method of treating benign prostatic hyperplasia in a mammal, wherein the method comprises administering to the mammal in need thereof a therapeutically effective amount of a compound of claim 1, claim 3 or claim 5, as a stereoisomer, enantiomer, tautomer thereof or mixtures thereof; or a pharmaceutically acceptable salt, solvate or prodrug thereof.

18. A method of treating pruritis in a mammal, wherein the method comprises administering to the mammal in need thereof a therapeutically effective amount of a compound of claim 1, claim 3 or claim 5, as a stereoisomer, enantiomer, tautomer thereof or mixtures thereof; or a pharmaceutically acceptable salt, solvate or prodrug thereof.

19. A method of treating cancer in a mammal, wherein the methods comprise administering to the mammal in need thereof a therapeutically effective amount of a compound of claim 1, claim 3 or claim 5, as a stereoisomer, enantiomer, tautomer thereof or mixtures thereof; or a pharmaceutically acceptable salt, solvate or prodrug thereof.
